(19) United States
(12) Patent Application Publication
(10) Pub. No.: US 2020/0335219 A1
(43) Pub. Date: Oct. 22, 2020

(54) SYSTEMS AND METHODS FOR PROVIDING PERSONALIZED PROGNOSTIC PROFILES

(71) Applicant: PointRight Inc., Cambridge, MA (US)

(72) Inventor: Barry S. Fogel, Cambridge, MA (US)

(21) Appl. No.: 16/715,628

(22) Filed: Dec. 16, 2019

Related U.S. Application Data
(63) Continuation of application No. 14/974,236, filed on Dec. 18, 2015, now abandoned.

(51) Int. Cl. G16H 50/30; G16H 50/70; G16H 10/60; G16H 50/20

(52) U.S. Cl. CPC G16H 50/30; G16H 50/20; G16H 10/60; G16H 50/70

(57) ABSTRACT

Systems and methods are presented for providing personalized prognostic profiles. Personalized prognostic profiles corresponding to the person of interest are generated and include: a personalized prognostic graph showing the historical outcomes of a matched population that is a subset of a reference population over a display interval; a widget containing identifying information about the index patient, indicating the forced match variables, the time interval(s) of interest, and the treatment of interest if applicable, and one or more supplemental widgets providing further information concerning the index patient's clinical status, care received, care plans, care preferences, or issues related to illness-related clinical or personal concerns of the index patient.

### RELATED APPLICATIONS

This patent application is a Continuation Application of U.S. patent application Ser. No. 14/974,236, filed on Dec. 18, 2015, the contents of which are incorporated herein by reference in their entirety.

### FIELD OF THE INVENTION

The present invention generally relates to systems and methods for providing personalized prognostic profiles, and more specifically to generating and providing personalized prognostic graphs.

### BACKGROUND

Thoughtful and ethical end-of-life care planning, with palliation of symptoms and avoidance of invasive, expensive and ultimately futile attempts to prolong life, is gaining importance as a concern for the medical profession and the general public. In the healthcare system as a whole, hundreds of billions of dollars are spent on care in the last six months of life, much of which neither prolongs life nor relieves suffering. At the same time, there are ethical concerns about pressuring patients to forgo treatment for financial reasons alone, and regulatory concerns about the abuse of Medicare hospice care insurance benefits for patients who do not have a terminal prognosis. Addressing these concerns requires improvement in physicians' abilities to make a prognosis for a patient's survival, and improvement in their ability to communicate a credible prognosis to patients, families, and other stakeholders. An accurate prognosis is the foundation of a rational discussion of advance care plans, including decisions to forgo certain treatments. Similarly, accurate estimation and description of the likely effects of treatments can aid greatly in shared physician-patient decision-making in literally life-and-death situations.

Physicians typically make a prognosis regarding a patient's survival by synthesizing and weighing several kinds of information, including: (1) published articles and other texts concerning the outcomes of particular diseases and conditions that afflict the patient; (2) publications about mortality prognoses in general, including predictive algorithms, formulas, and rating scales; (3) history reported by the patient or about the patient (e.g., by family, nurses, or other observers); (4) findings on examination of the patient; (5) laboratory test results, imaging reports, and other objective data; (6) review of medical records; and (7) the physician's experience with similar patients in similar settings.

Physicians convey prognoses to patients, families, and other stakeholders in a variety of ways, sometimes quantitative and probabilistic and sometimes qualitative and subjective. Frequently, a physician is reluctant to make a definite statement about a patient's survival prognosis because of his or her own uncertainty about it. This may be especially true when a patient has a combination of diseases and conditions affecting life expectancy rather than a single terminal disease. As a result, advance care planning, appropriate palliative care, and meaningful shared decision-making may not happen in a timely way, or happen at all. That is, timely care planning is negatively affected by, for example: (1) a prognosis for survival not being discussed until a few days or a few weeks before a patient dies; (2) a patient or a patient's family not understanding the prognosis as explained by the physician, or not remembering it accurately when they consider treatment options; (3) a patient or patient's family not finding the prognosis credible, or fearing that there is pressure to forgo treatment for an inappropriate reason; (4) advance care planning and other end-of-life considerations not being brought up and discussed concurrently with the conveying of the prognosis; (5) disagreement among various people close to the patient, including in many cases disagreement regarding the meaning, precision and/or validity of the medical prognosis; and (6) patients or their families not fully understanding options for further treatment, or not having a realistic view of what outcomes might be expected from specific potential treatments.

Thus there is a need for systems and methods that can, among other things: improve the timeliness and accuracy of survival prognoses for a wide range of patients, including those with multiple diseases and conditions; improve the comprehensibility and credibility of survival prognoses; improve the communication of survival prognoses to patients, families and other interested parties; assess the effect of various treatment options on the survival prognosis, and link the communication of a prognosis with the consideration of palliative care, advance directives concerning life-sustaining treatments, and other end-of-life issues.

### SUMMARY

The example embodiments presented herein are directed to systems and methods for providing personalized prognostic profiles.

In some example embodiments, a system is provided for providing personalized prognostic profiles, comprising: at least one memory operable to store a reference database associated with a plurality of individuals (e.g., a reference population), the reference database comprising a specified set of demographic and clinical information associated with each of the plurality of individuals; a processor communicatively coupled to the at least one memory, the processor being operable to: receive, over a network, from a client computing device, information associated with an index patient (e.g., person of interest) (e.g., a structured clinical assessment; an extract from an electronic medical record; laboratory or imaging data; genomic or proteomic data; or data transmitted from a mobile device, or, alternatively, an indicator of the location of such information within a designated database, wherein each data item (1) in the information associated with the index patient, and (2) in the stored demographic and clinical information associated with each of the plurality of individuals, that can vary over time is linked to a corresponding date or time interval; receive, over the network, from a client computing device, a first request to generate a personalized prognostic profile corresponding to the index patient, the first request comprising: (1) a binary clinical outcome of interest; (2) a display interval indicating the time interval that will be covered by the personalized prognostic profile; (3) either (a) two or more time intervals of interest that differ from each other and are each no greater than the display interval, or (b) one or more time periods of interest and a treatment of interest, and (4) forced match variables, wherein the forced match variables comprise one or more clinical or demographic items used to define a proper subset of the plurality of individuals, by requiring that every person in the subset exactly match the index patient on each of the forced match variables, wherein, if there are two or more time intervals of interest, each of the plurality of time intervals of interest is associated with a corresponding priority level with respect to the other time intervals; and generate a personalized prognostic profile corresponding to the person of interest, the personalized prognostic profile comprising: a personalized prognostic graph showing the historical outcomes of a matched population that is a subset of the reference population over the display interval, a necessarily included widget containing identifying information about the index patient, indicating the forced match variables, the time interval(s) of interest, and the treatment of interest if applicable, and, optionally, one or more supplemental widgets providing further information concerning the index patient's clinical status, care received, care plans, care preferences, or issues related to illness-related clinical or personal concerns of the index patient, wherein: (1) the matched population is a subset of the plurality of individuals (e.g., reference population) in which every person exactly matches the index patient on each of the forced match variables and a further property that an estimated probability that the outcome of interest at one or more user-specified time intervals following a starting point is within a predetermined interval of the estimated probability of the outcome of interest for the index patient, wherein predictions are made using predictive models developed on a subset of the reference population that exactly matches the patient on each of the forced match variables, and wherein the starting point is a point at which there are valid values in the reference database for all of the forced match variables and there are known or imputable values of all variables used in the predictive models; (2) the personalized prognostic graph shows actual outcomes over the display interval after a starting point for all members of the matched population; (3) the necessarily included widget comprises sufficient information for users to know facets of the prognostic profile that were personalized (e.g., the forced match variables, the time interval(s) selected and their order of priority, and the treatment of interest selected for consideration, if any); and (4) the one or more supplemental widgets comprising checklists of issues for consideration (e.g., by the index patient, family members or significant others of the patient, clinicians, and/or other professionals involved in caring for or serving the index patient).

In some example embodiments, the processor is further operable to: identify a forced match subset of individuals from among the plurality of individuals associated with the reference database, wherein the each of the individuals in the forced match subset exactly matches the index patient on each of the forced match variables received in the first request; generate a first outcome predictive model on the forced match subset, or on a randomly selected subset of the forced match subset, that predicts the occurrence of the outcome of interest over the first specified time interval of interest, wherein the first outcome predictive model utilizes a subset of the variables in the reference database as predictors, and may utilize any predictive modeling formula or algorithm that is generally recognized as applicable for clinical prediction (e.g., logistic regression, polynomial regression, spline regression, decision tree, neural net, boosted regression, lasso regression, boosted tree, cluster analysis, random forest, and support vector machine methodologies); calculate, using the first outcome predictive model, the probability of the occurrence of the outcome of interest at a first specified time interval (1) for the index patient, and (2) for each of the individuals in the forced match subset or on the complement of the randomly selected subset used to generate the first outcome predictive model; identify, from among the individuals in the forced match subset or on the complement of the randomly selected subset used to generate the predictive model, a first nested subset of individuals in which every member of the subset has, according to the first predictive model, an estimated probability of the occurrence of the outcome of interest within the first specified time interval of interest that is within a given predetermined interval of the probability of the occurrence of the outcome of interest of the person of interest; and repeat the process of generating the predictive model for of the outcome of interest and nested subset selection for each of any additional of the time intervals, in order of priority, creating a new predictive model at each repetition that is estimated on the nested subset created in the previous step in the iterative process, or on a randomly selected subset of the nested subset, applied to the index patient and to all members of the nested subset or to all members of the complement of the randomly selected subset used for model generation, and used to select a nested subset of the nested subsets in which the estimated probability of occurrence of the outcome of interest within the time interval of interest lies within a predetermined interval of the estimated probability for the index patient; if a treatment has been specified that was received by a proper subset of individuals in the reference database, estimate on the final nested subset, or on a randomly selected subset of the final nested subset generated thus far in the process, a predictive model (i.e., a propensity model) for the receipt of the treatment; apply that predictive model to the index patient and to all patients in the final nested subset generated thus far in the process, or to the complement of the subset used to generate the propensity model; create a nested subset of patients in which each member of the subset has an estimated probability of receiving the treatment that lies within a predetermined interval of the estimated probability for the index patient; designate the nested subset that is the final result of the iterative process as the matched population; and display the occurrence of the outcome of interest in the matched population over the display interval or shorter interval if the latter is specified in the user's request, with the outcome of the treated and the untreated patients displayed separately if a treatment was specified by the user.

In some example embodiments, the last identified nested subset is the matched population. In other example embodiments, the matched population is a subset of the last identified nested subset selected by requiring a closer match on the estimated probability of the outcome of interest over a particular interval of interest than was utilized in creating the nested subset used in the iterative modeling process.

In some example embodiments, the information associated with each of the plurality of individuals comprises a binary variable indicating whether a particular treatment was given or a procedure was performed at a given time or within a given time interval following the starting point, for one or more specific treatments, wherein the first request further comprises a treatment of interest that might be a clinical consideration for the index patient but that the index patient had not received as of the starting point for the personalized prognostic profile; wherein the processor is further operable to: generate a treatment predictive model (i.e., propensity model) based on one or more of the received potential predictor variables associated with the person of interest and the potential predictive variables associated with each of the individuals in the forced match subset, and estimated on the matched population or on a randomly selected subset of it; calculate, using the treatment predictive model, the probability of the occurrence of the treatment of interest for (1) the index patient, and (2) each of the individuals in the last identified subsequent nested subset or on the complement of the randomly selected subset used for creating the propensity model; identify a propensity matched subset of individuals from among the individuals in the last identified nested subset, wherein the estimated likelihood of the occurrence of the procedure of interest for each of the individuals in the propensity matched subset is within a given predetermined interval of the index patient's estimated probability of receiving the treatment of interest; and identify, based on the binary treatment data in the stored information of the individuals in the propensity matched subset, a treatment-true subset and a treatment-false subset, wherein the treatment-true subset comprises individuals, from among the individuals in the propensity matched subset who actually received the treatment of interest within the time interval specified in the definition of the binary treatment data interest, and wherein the treatment-false subset comprises individuals, from among the individuals in the propensity matched subset who did not receive the treatment of interest within the time interval specified in the definition of the binary treatment data, and wherein the personalized prognostic graph indicates the occurrence over time of the outcome of interest for the individuals in the treatment-true subset independently from the occurrence of the outcome of interest for the individuals in the treatment-false subset.

In some example embodiments, the propensity matched subset is the matched population.

In some example embodiments, the information associated with each of the plurality of individuals comprises: (1) independent variables comprising demographic data (e.g., age, gender, race) and clinical data (e.g., diagnoses, conditions, functional status, mental status, behavioral status, physiologic measures, medications, procedures, and other data extracted from electronic medical records); and (2) outcome data (e.g., date, yes/no flag) indicating the occurrence of one or more binary outcomes (e.g., death, loss or gain of physical or mental function, medical event (e.g., fall, stroke)). In typical embodiments death versus survival of the patient is included among the binary outcomes.

In some example embodiments, the processor is further operable to: identify, for each of a plurality of system-specified time periods, one or more candidate predictor variables from among the types of the independent variables stored in the reference database, the candidate predictor variables comprising demographic variables, variables related to the context of treatment, and clinical variables identified based on a measure of the relationship between the variable and one or more binary outcomes, wherein at least one of the forced match predictor variables is a demographic variable and one or more clinical forced match predictor variables are selected from among the one or more candidate clinical predictor variables.

In some example embodiments, at least a portion of the one or more candidate predictor variables are associated with a respective assessment date or time interval indicating a date or time interval on which a given independent variable was measured.

In some example embodiments, the reference database is generated from data retrieved from external third-party systems.

In some example embodiments, the reference database is updated on a regular basis with a maximum interval between updates.

In some example embodiments, the criteria for forced matches includes a requirement that the starting dates for measuring the outcomes of interest in the matched population be no earlier than a particular date, to ensure that the personalized prognostic profile reflects the outcomes of contemporary practice.

In some example embodiments, the processor is further operable to cause to display, at least at one of the plurality of client computing devices, the personalized prognostic profile.

In some example embodiments, each of the generated outcome predictive model is stored in the at least one memory.

In some example embodiments, the contextual data comprises one or more of (1) a listing of the candidate predictor variables having the strongest effect on the outcome predictive models; (2) measures of the accuracy of the predictive models used to select the nested subsets; (3) an estimated probability that the index patient would receive the specified treatment of interest, as determined by the predictive model for receiving that treatment; (4) source(s) of the data in the reference database; (5) a date on which the reference database was last updated; and (6) an earliest starting date for measuring the occurrence over time of the outcome of interest, for all individuals in the matched population.

In some example embodiments, the binary clinical outcome of interest is death, wherein one or more of supplemental widgets comprises one or widgets concerned with end-of-life care, such as palliative care for symptom or advance directives for life-sustaining treatments.

In some example embodiments, a method is provided for providing personalized prognostic profiles, comprising: providing at least one memory operable to store a reference database associated with a plurality of individuals (i.e., a reference population), the reference database comprising a specified set of demographic and clinical information associated with each of the plurality of individuals; receiving, over a network, from a client computing device, information associated with an index patient (more generally, a person of interest) (e.g., a structured clinical assessment; an extract from an electronic medical record; laboratory or imaging data; genomic or proteomic data; or health-related data transmitted from a mobile device), or, alternatively, an indicator of the location of such information within a designated database, wherein each data item (1) in the information associated with the index patient, and (2) in the stored demographic and clinical information associated with each of the plurality of individuals, that can vary over time is linked to a corresponding date or time interval; receiving, over the network, from a client computing device, a first request to generate a personalized prognostic profile corresponding to the index patient, the first request comprising: (1) a binary clinical outcome of interest; (2) a personal time frame (display interval) indicating the time interval that will be covered by the personalized prognostic profile; (3) either (a) two or more time intervals of interest that differ from each other and are each no greater than the display interval, or (b) one or more time periods of interest and a treatment of interest, and (4) forced match variables, wherein the forced match variables comprise at least one demographic variable and one or more clinical items used to define a proper subset of the plurality of individuals (i.e., the forced match population), by requiring that every person in the subset exactly match the index patient on each of the forced match variables, wherein, if there are two or more time intervals of interest, each of the plurality of time intervals of interest is associated with a corresponding priority level with respect to the other time intervals; and generating a personalized prognostic profile corresponding to the person of interest, the personalized prognostic profile comprising: a personalized prognostic graph showing the historical outcomes of a matched population that is a subset of the reference population over the display interval, a necessarily included widget containing identifying information about the index patient, indicating the forced match variables, the time interval(s) of interest, and the treatment of interest if applicable, and, optionally, one or more supplemental widgets providing further information concerning the index patient's clinical status, care received, care plans, care preferences, or issues related to illness-related clinical or personal concerns of the index patient, wherein: (1) the matched population is a subset of the plurality of individuals (i.e., reference population) in which every person matches the index patient exactly on all of the forced match variables and a further property that an estimated probability of the occurrence of outcome of interest during one or more user-specified time intervals following a starting point is within a preset interval of the estimated probability for the index patient of the outcome of interest occurring during that interval, wherein predictions are made using predictive models developed on a subset of the reference population in which each member matches the patient exactly on all of the forced match variables, and wherein the starting point is a point in which there are valid values in the reference database for all of the forced match variables and known or imputable values of all variables used in the predictive models, employing an iterative process in which the predictive model at each step is generated, validated, and applied to subsets of the set of patients created at the previous step, culminating in the selection of a matched population in which each member matches the index patient exactly on all of the forced match variables and each member's estimated probability on each of two or more predictive models falls within a specified interval (e.g., defined by absolute differences, by percentages, or by numbers of patients) of the estimated probability for the index patient; (2) the personalized prognostic graph shows actual outcomes over the display interval after a starting point for all members of the matched population; (3) the necessarily included widget comprises sufficient information for users to know facets of the prognostic profile that were personalized (e.g., the forced match variables, the time interval(s) selected and their order of priority, and the treatment of interest selected for consideration, if any); and (4) the one or more supplemental widgets comprise checklists of issues for consideration by the user or other individuals concerned with the index patient's care and/or outcomes (e.g., by the index patient, family members or significant others of the patient, clinicians, and/or other professionals involved in caring for or serving the index patient).

In some example embodiments, a system provides personalized prognostic profiles, comprising: at least one memory operable to store a reference database associated with a plurality of individuals (i.e., reference population), the reference database comprising information associated with each of the plurality of individuals; a processor communicatively coupled to the at least one memory, the processor being operable to: receive, over a network, from one of a plurality of client computing devices, information associated with a person of interest (e.g., structured clinical assessment, medical record data, output of mobile devices); receive, over the network, from one of the plurality of client computing devices, a first request to generate a personalized prognostic profile corresponding to the person of interest (e.g., index patient), the first request comprising (1) an outcome of interest, (2) a starting point, (3) a time frame, 4) a plurality of time periods of interest, and (5) one or more forced match predictor variables, wherein each of the plurality of time periods of interest is associated with a corresponding priority level with respect to the other time periods (e.g., time period with first priority, time period with last priority); and generate the personalized prognostic profile corresponding to the person of interest, the personalized prognostic profile comprising one or more of: (1) a personalized prognostic graph indicating the occurrence of the outcome of interest within a matched population, the occurrence of the outcome of interest being measured at least at each of the plurality of time periods of interest; (2) one or more supplemental widgets (e.g., checklist widgets) for managing a plurality of issues associated with the outcome of interest; and (3) contextual data associated with one or more of the person of interest and the personalized prognostic graph widget.

In some example embodiments, the processor is further operable to: identify a forced match subset of individuals from among the plurality of individuals associated with the reference database, wherein the information associated with the individuals in the forced match subset matches the one or more forced match predictor variables received in the first request; generate an outcome predictive model based on one or more of the received information associated with the person of interest and the information associated with each of the individuals in the forced match subset; calculate, using the outcome predictive model, the probability of the occurrence of the outcome of interest at a first time period for the person of interest, wherein the first time period is associated with the highest priority level; identify, from among the individuals in the forced match subset, a first nested subset of individuals with an estimated probability of the occurrence of the outcome of interest that is within a given predetermined interval of the probability of the occurrence of the outcome of interest of the person of interest; and identify subsequent nested subsets of individuals for each of the remaining time periods based on calculated probabilities of the occurrence of the outcome of interest at each time period for the person of interest, wherein the estimated probabilities of the occurrence of the outcome of interest are calculated using the outcome predictive model, wherein, for each of the subsequent nested subsets, the probability of the occurrence of the outcome of interest of the individuals for a given subsequent nested subset is within a given predetermined interval around the probability of the occurrence of the outcome of interest of the person of interest, wherein the subsequent nested subsets are iteratively identified, ending with the time period associated with the lowest priority level, wherein at each step of the iterative process, if the nested subset contains enough patients, one randomly selected subset of the nested subset may be used to generate and validate the predictive model, and the source of the individuals with predicted probabilities within a predetermined interval around the predicted probability for the index patient will then be the complement of the subset used to generate the predictive model.

In some example embodiments, the last identified subsequent nested subset is the matched population.

In some example embodiments, the information associated with each of the plurality of individuals comprises binary treatment data (e.g., date, yes/no flag) indicating, for each respective individual, the occurrence, within a specified interval following the starting point, of a specific treatment or combination of treatments, wherein the first request further comprises a treatment or combinations of treatments of interest, wherein the processor is further operable to: generate a treatment predictive model (e.g., propensity model) based on the received information associated with the person of interest and the information associated with each of the individuals a subset of the forced match population that was created at the previous step in the iterative process or on a randomly selected subset of that subset; calculate, using the propensity model, the probability of the occurrence of the treatment of interest for (1) the person of interest, and (2) each of the individuals in the subset of the forced match population created in the previous step in the iterative process, or, alternatively in the complement of the randomly selected subset of that subset that was used to generate and validate the propensity model; identify a propensity matched subset of individuals from among the individuals in the last identified subsequent nested subset or from among the individuals in the complement of the subset of the latter that was used to generate and validate the propensity model, wherein the probability of the occurrence of the treatment of interest for each of the individuals in the propensity matched subset is within a given predetermined interval around the estimated propensity of the occurrence of the treatment of interest for the index patient; and identify, based on the binary treatment data in the stored information of the individuals in the propensity matched subset, procedure treatment-true subset and a treatment-false subset, wherein the treatment-true subset comprises individuals, from among the individuals in the procedure match subset, who actually had the treatment interest, and wherein the treatment-false subset comprises individuals, from among the individuals in the procedure match subset who did not have the treatment of interest, and wherein the personalized prognostic graph indicates the occurrence of the outcome of interest for the individuals in the treatment-true subset independently from the occurrence of the outcome of interest for the individuals in the treatment-false subset.

In some example embodiments, the propensity matched subset is the matched population.

In some example embodiments, the information associated with each of the plurality of individuals comprises: (1) independent variables comprising demographic data (e.g., age, gender, race) and clinical data (e.g., diagnoses, conditions, functional status, mental status, behavioral status, physiologic measures, medications, procedures, image analysis, physiologic measurements); and (2) outcome data (e.g., date, yes/no flag) indicating the occurrence of one or more binary outcomes (e.g., death, loss or gain of physical or mental function, medical event (e.g., fall, stroke)).

In some example embodiments, the processor is further operable to: identify, for each of a plurality of system-specified time periods, one or more candidate predictor variables from among the types of the independent variables stored in the reference database, the candidate predictor variables being identified based on a measure of the relationship between the types of the independent variables and one of the one or more binary outcomes, wherein the one or more forced match predictor variables are selected from among the available demographic variables and one or more of the candidate predictor variables.

In some example embodiments, at least a portion of the one or more candidate predictor variables are associated with a respective assessment date or assessment interval indicating a date on which or interval over which a given independent variable was measured.

In some example embodiments, the reference database is generated from data retrieved from external third-party systems.

In some example embodiments, the reference database is validated in response to the receiving of the request to generate a personalized prognostic profile, to ensure that the reference database contains sufficiently up-to-date information to support the user's purposes with respect to the personalized prognostic profile (e.g., information reflecting updated information in the external third-party systems).

In some example embodiments, the processor is further operable to cause to display, at least at one of the plurality of client computing devices, the personalized prognostic profile.

In some example embodiments, each of the generated outcome predictive model is stored in the at least one memory.

In some example embodiments, the contextual data comprises one or more of: (1) the number of candidate predictor variables and/or types of candidate predictor variables used in the outcome predictive models and, if applicable, the treatment predictive model (propensity model); (2) one or more candidate predictor variables having the strongest effect on the outcome predictive models and/or treatment predictive model; (3) the type of model(s) used for outcome prediction; (4) a measure of model performance (e.g., predictive value, receiver operating characteristic) for one of more of the predictive models used in creating the matched population; (5) the source(s) of the data used in creating the matched population; and (6) the currency of the data used to create the matched population, specifically the earliest starting point for the data, and the date on which the data were last updated.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

The foregoing and other objects, aspects, features, and advantages of the present disclosure will become more apparent and better understood by referring to the following description taken in conjunction with the accompanying drawings, in which:

FIG. 1 is a system for providing personalized prognostic profiles, according to an exemplary embodiment.

FIG. 2 illustrates a flowchart for providing personalized prognostic profiles, according to an exemplary embodiment.

FIG. 3A illustrates a personalized prognostic profile, according to an exemplary embodiment.

FIG. 3B illustrates a personalized prognostic profile request page for receiving user inputs, according to an exemplary embodiment.

FIG. 3C illustrates a personalized prognostic profile including a graph illustrating a comparison of survival of patients having versus not having received a specified treatment (e.g., treated vs. untreated patients), according to an exemplary embodiment.

FIG. 3D simultaneously displays the personalized prognostic graphs (in this case survival graphs) generated for one specific, exemplary patient with heart failure, under four different conditions (see Example 2 for details), illustrating a difference of great practical significance in the graphs using a generic predictive model and those generated by the system of the present invention. The X axis represents time (e.g., number of days, and the Y axis represents the probability of survival).

FIG. 4 is a block diagram of an example network environment for use in the methods and systems for providing personalized mortality prognostic reports, according to an exemplary embodiment.

FIG. 5 is a block diagram of an example computing device and an example mobile computing device, for use in illustrative embodiments of the invention.

### DETAILED DESCRIPTION

### Definitions

In order for the present disclosure to be more readily understood, certain terms are first defined below. Additional definitions for the following terms and other terms are set forth throughout the specification.

“Outcome of interest”: the term “outcome of interest,” as used herein, refers to a binary outcome of clinical interest; in the embodiment described this outcome is death. In the most general application of the technology described herein, the binary outcome may be a non-medical outcome, for example a financial or a social outcome.

“Reference database”: the term, “reference database,” as used herein, refers to a collection of demographic, clinical, and other person-specific data on a large number of patients that comprises for each patient the values of numerous variables the many potential predictors of the outcome of interest, with each such variable linked explicitly or implicitly to a time point (date and possibly time of day) or a time interval, and either the date and possibly time of occurrence of the outcome of interest or a time interval within which the outcome occurred, or a time interval during which the outcome of interest definitely did not occur.

“Starting point”: the term, “starting point,” as used herein, refers to a point in time at which the current values of many variables potentially predictive of the outcome of interest are known or can be accurately inferred or usefully imputed from known values of other potentially predictive variables.

“System time frame”: the term, “system time frame,” as used herein, refers to a time interval over which for every patient in the reference database there is a starting point at which many potentially predictive variables are known and a time interval at least as long as the time frame over which it is known definitely whether or not the outcome of interest occurred.

“Index patient”: the term, “index patient,” as used herein, refers to an individual patient with an illness or condition potentially affecting life expectancy, whose expected survival is of interest to a user, or, in the more general case, an individual patient at risk for a binary clinical outcome that might or might not occur within the system time frame. The values of a number of potentially predictive variables are known for the index patient at a particular point in time that is the starting point for that specific patient.

“User”: the term, “user,” as used herein, refers to a person legitimately concerned with the occurrence of the outcome of interest in the index patient, in the embodiment described herein the user is concerned with the survival or death of the index patient. The user can be without limitation the patient, a family member or friend, a health professional, a lawyer or a financial professional. If the user is not the index patient, the user is presumed to have a legal right to see clinical information about the index patient.

“Personal starting point”: the term, “personal starting point,” as used herein, refers to a point in time at which the values a number of potentially predictive variables are known (or can be accurately inferred or usefully imputed) for the index patient.

“Personal time frame”: the term, “personal time frame,” as used herein, refers to a time interval beginning with the personal starting point over which the occurrence of outcome of interest to the patient of interest is of concern to the user. The personal time frame is equal to or shorter than the system time frame. The personal time frame is the display interval for the personalized prognostic graph. The terms “personal time frame” and “display interval”, as used at different points herein, are intended to represent the same time period.

“Matched population”: the term, “matched population,” as used herein, refers to a set of patients that is a subset of the patients represented in the reference database that has been matched with the patient of interest utilizing the methodology described herein. For each member of the matched population there is an associated starting point at which the potentially predictive variables are known and after which the occurrence of the outcome of interest is known for the system time frame, and thus known for the personal time frame, which is no greater than the system time frame.

“Forced match variables”: the term, “forced match variables,” as used herein, refers to a set of potentially predictive variables, always including one or more demographic factors, on which every member of the matched population has exactly the same values as the index patient.

“Primary personally important time interval”: the term, “primary personally important time interval,” as used herein, refers to a time interval over which the survival of the patient (more generally, the occurrence of the outcome of interest) is of particular interest to the user. The primary personally important time interval will be no greater than the display interval, e.g., the personal time frame.

“Second (or nth) personally important time interval”: the term, “second (or nth) personally important time interval,” as used herein, refers to a time interval over which the survival of the patient (more generally, the occurrence of the outcome of interest) is of particular interest to the user, though less so than the primary personally important time interval (or, more generally, less than the (n−1)th personally important time interval. All of these non-primary personally important time intervals are no greater than the display interval.

“Potential treatment time frame”: the term, “potential treatment time frame,” as used herein, refers to a time interval shorter than the personal time frame over which a patient might or might not receive a treatment of interest.

“Treatment of interest”: the term, “treatment of interest,” as used herein, refers to a treatment that the patient might or might not receive during the potential treatment time frame; the decision to give the treatment to the index patient is of interest to the user. It is implicit that the treatment of interest potentially affects the outcome of interest, either intentionally or incidentally.

“Survival graph”, “survival curve”, “prognostic graph”, and “prognostic curve”: these terms, as used herein, refer to an x-y plot based on historical data on a given population that shows for numerous time points over a time frame following a starting point the proportion of the population that remained alive at that point (more generally, had not had the outcome of interest as of that point). The proportion is 100% at the starting point. A corresponding “mortality graph” or “mortality curve” for the same data would display for numerous time points after a starting point the proportion of the population that had died by that point (more generally, had the outcome of interest by that point).

“Expected survival graph” or “expected survival curve”: this term “expected survival curve,” as used herein, refers to an x-y plot based on a predictive model that indicates, for several time points in a time interval after a starting point, the percentage probability that a particular patient will be alive at that time point (i.e., will not die over the interval between the starting point and that time point). More generally, when the outcome of interest is not death, the corresponding curve may be referred to as an “expected outcome curve”. Note that the expected survival graph may be the actual (historical) survival graph for population of patients that, based on assessment with a predictive model, has a similar expected outcome to that of a patient of interest.

“Probability of survival”: the term “probability of survival,” as used herein, refers to an estimated probability that a particular patient will survive until a specified time after a starting point, or the estimated percentage of a particular population that will be alive at a specified time after a starting point. A customary way for a physician to describe a prognosis is to give a probability of survival over a single specified time interval (i.e., survival at least until a specified time point). Less often, a physician will display an expected survival curve and explain it to the patient or other interested party. In the system described herein the user (who may be a physician or a patient) sees a population survival curve for a matched population, where the matched population is so similar to the index patient, the size of the matched population is sufficient, and the data on the matched population it so recent and so similar in its context that the user finds the observed (historical) survival of the matched population to be a credible expected survival curve for the patient. The personalized survival graph is in a typical embodiment a Kaplan-Meier survival curve. However, there are other ways to represent the same information, such as:
The number of patients in the matched population can be indicated on either the x-axis or the y-axis, with the time from the starting point represented on the other axis.
(ii) The number of patients in the matched population can be indicated as an absolute number or as a percentage.

The relationship of time and survival (more generally, of time and the occurrence of the outcome of interest) can be shown as a curve, a bar chart, a column chart, or a pattern of icons (e.g., icons representing 100 patients).

### System

The example embodiments described herein are directed to systems and methods for providing personalized prognostic profiles.

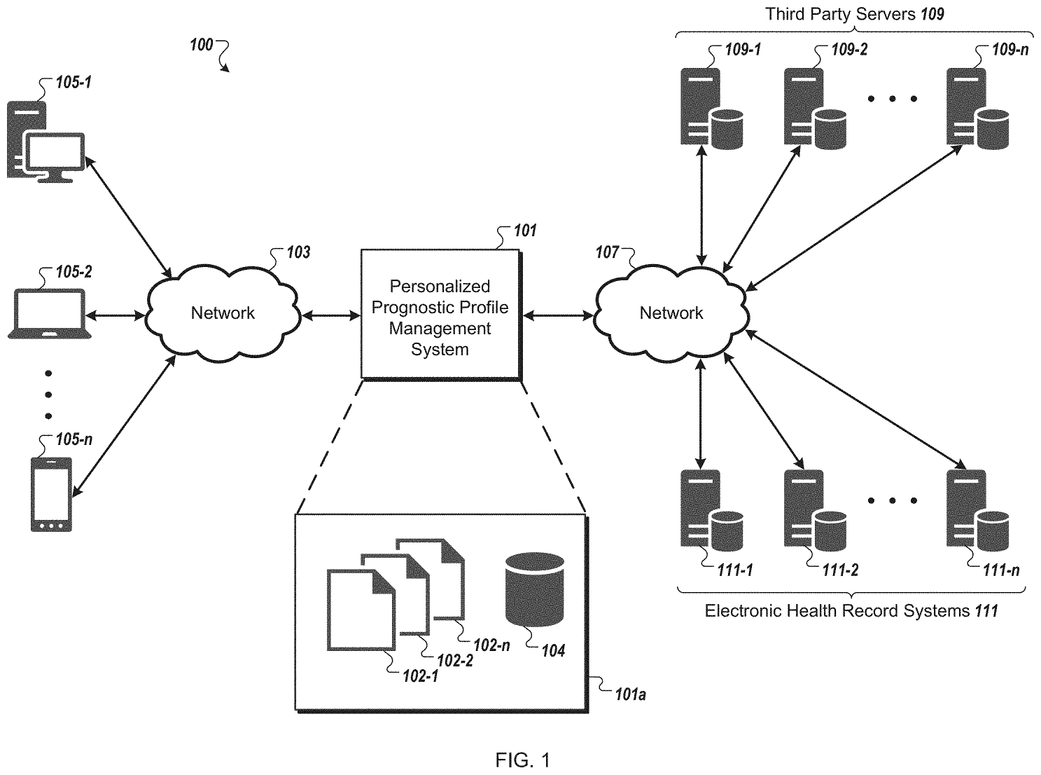

FIG. 1 illustrates a system 100 for providing personalized prognostic profiles, according to an exemplary embodiment. As shown in FIG. 1, system 100 includes a personalized prognostic profiles management system 101. The system 101 is used to generate, store, track, and manage personalized prognostic profiles for users and/or patients. Personalized prognostic profiles, including the generation thereof, are described in more detail below with reference to FIGS. 1-3C.

The personalized prognostic profiles management system 101 may be or include one or more computing and/or electronic devices equipped with hardware (e.g., processor, storage means, display) and/or software. For example, the report management system 101 may include one or more servers such as database servers, file servers, mail servers, print servers, web servers, application servers, and the like. As illustrated in FIG. 1, the personalized prognostic profile management system 101 includes at least one memory 101a for storing personalized prognostic profiles, and/or data (e.g., health record data obtained from electronic health record systems (111), and/or data obtained from third party servers (109)), for example, in one or more reference databases.

That is, as illustrated in FIG. 1, the report management system 101 is communicatively coupled to third party servers 109-1, 109-2, . . . , 109-n (collectively “third party servers” and/or “109”) and electronic health record systems 111-1, 111-2, . . . , 111-n (collectively “electronic health record systems” and/or “111”), over a network 107. The network 107 may be a virtual private network (VPN), local area network (LAN), personal area network (PAN), wide area network (WAN), the Internet or the like. It should be understood that although the network 107 is illustrated as a single element in FIG. 1, the network 107 may include any number and combination of networks.

In some example embodiments, the servers 109 may be and/or include mortality databases such as the Social Security Administration Death Master File database, health plan or health system databases, state vital statistics offices databases, or other sources that capture dates of death, from private and/or government entities. The electronic health record systems 111 include health record data and/or health record databases managed and/or maintained by medical centers, hospitals, integrated care systems, or other health care providers; health plans, care management companies, insurance carriers or other payers and their information technology vendors; and government agencies.

A reference database may be generated and/or maintained using data obtained from multiple systems (e.g., mortality databases, electronic health records), by querying the data (e.g., automatic query) through a suitable application protocol interface (API). The data in the reference database are stored in a standardized (e.g., identically or substantially identically structured) form. If data acquired from contributing systems is not in an optimal standardized form, the standardized data required for the creation of personalized prognostic profiles is created from the data initially input to the database, via some combination of re-coding, application of business rules, data reduction, and structured inference. Natural language processing algorithms may be utilized to convert free text medical records into standardized data elements. Codes for diagnoses and procedures may be converted into categorical variables suitable for analysis; laboratory tests may be converted into standard units. Imaging data may be reduced to categories (e.g., fracture or no fracture”) or converted into quantitative data (e.g., measurement of tumor size or application or an ordinal scale of severity of joint degeneration). Moreover, in some example embodiments, health record data is input manually or automatically, by users and/or physicians into the report management system 101. For example, a physician may directly input health record data for a patient into the report management system (e.g., via a computing device). In another example, a patient may input health record data into the report management system, or may have information pushed from a mobile device (e.g., smart phone or wearable device) into the report management system. Input data is used to supplement and/or replace data stored in the system 101. In other embodiments the user may indicate the location of the patient's clinical and/or contextual data in an external database that can be queried by the system.

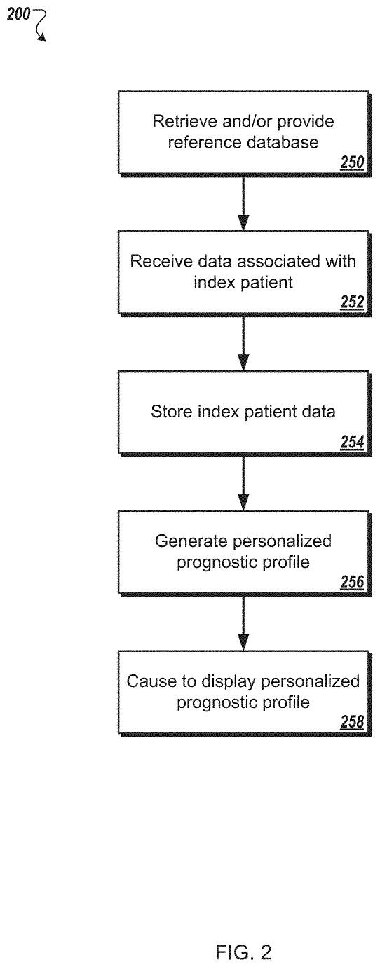

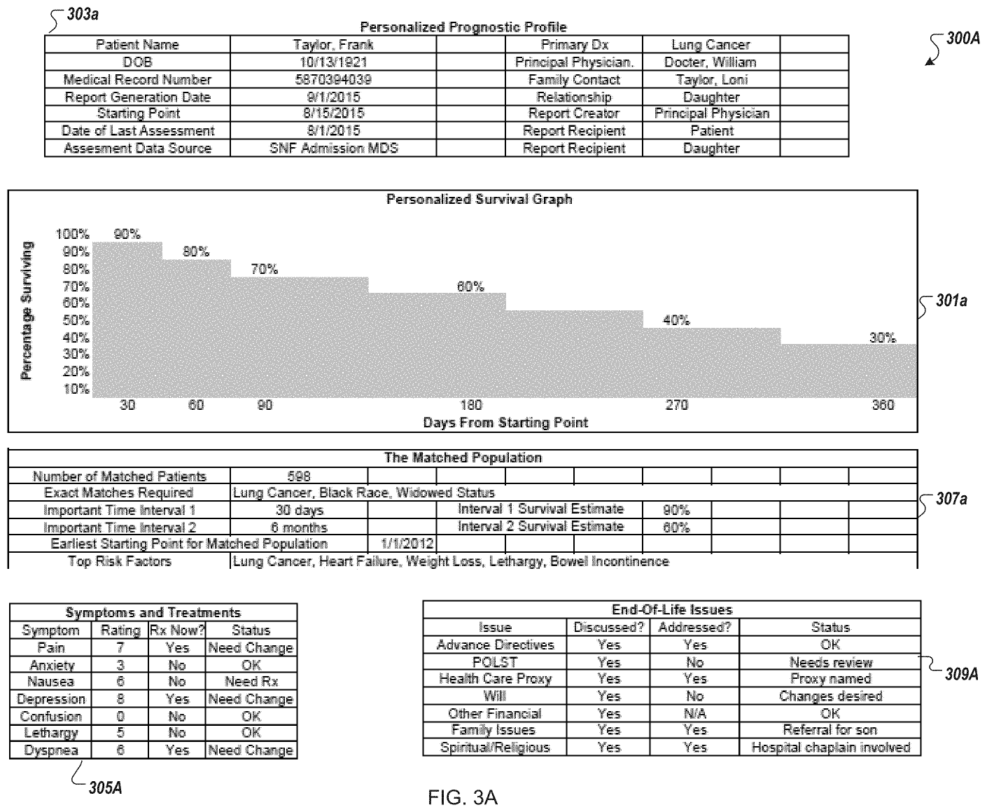

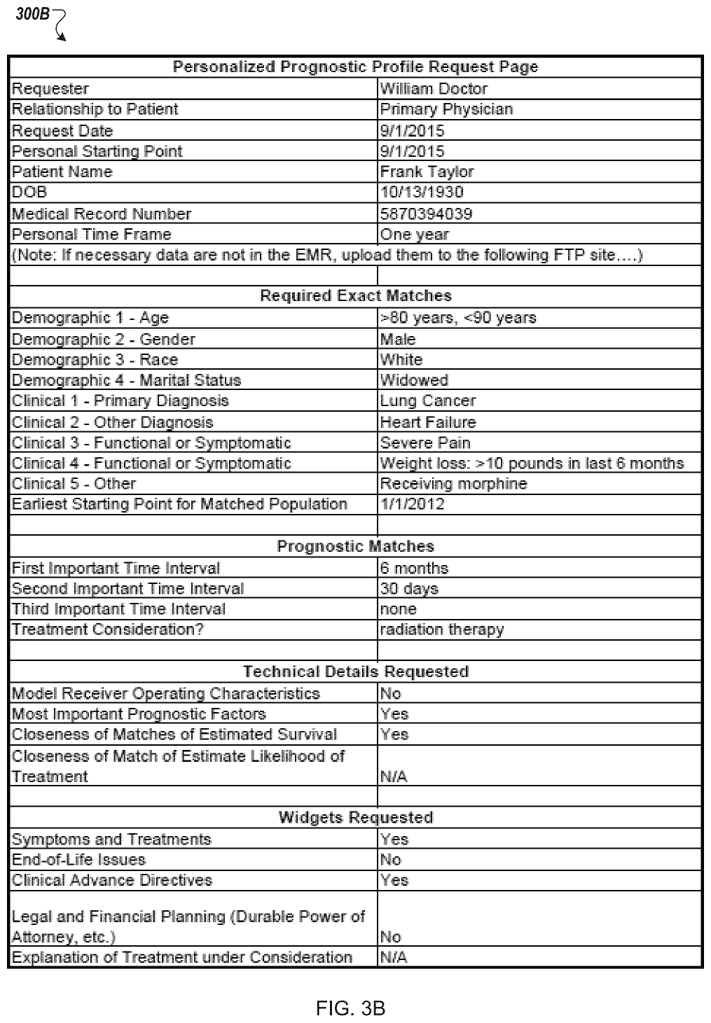

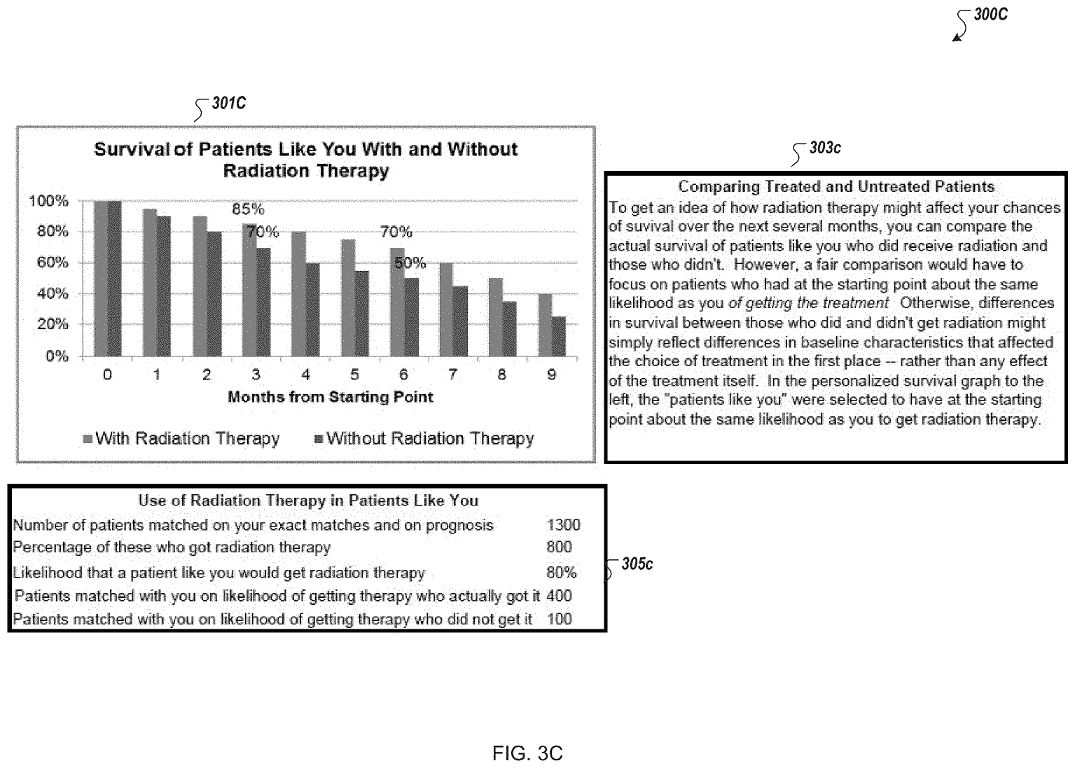

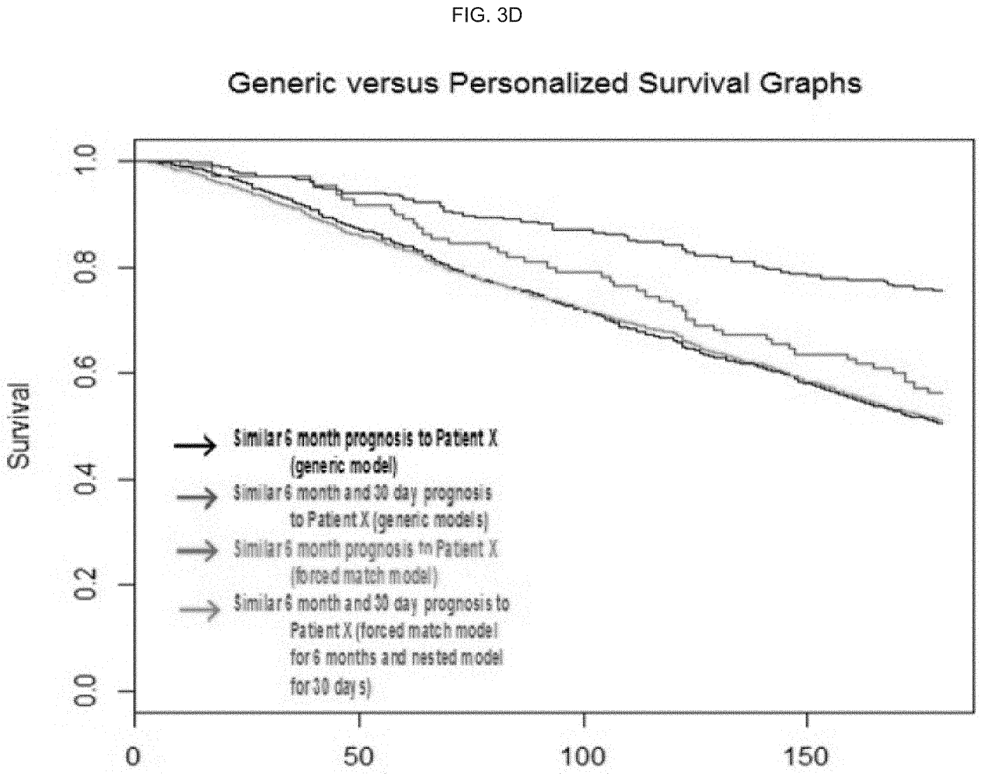

The one or more memories 101a of the report management system 101 may be used to store and/or personalized prognostic profiles (e.g., 102-1, 102-2, . . . , 102-n). As described in further detail below with reference to FIGS. 2 and 3A-3C, each personalized prognostic profile consists of a personalized prognostic graph and one or more widgets that comprise content linked to the content of the personalized prognostic graph, as described further below.

The report management system 101 is communicatively coupled to client computing devices 105-1, 105-2, . . . , 105-n (collectively “client computing devices” and/or “105”) over a network 103. The network 103 may be a virtual private network (VPN), local area network (LAN), personal area network (PAN), wide area network (WAN), the Internet and the like. It should be understood that although the network 103 is illustrated as a single element in FIG. 1, the network 103 may include any number and combination of networks.

Each of the client computing devices 105 may be operated, managed, and/or owned by a user. In some example embodiments, a user is a patient, family member, healthcare professional, lawyer, caregiver, financial professional, member of clergy, or the like, who is interested in the prognosis of the patient and is legally entitled to access information about that prognosis. The user, utilizing a client computing device, requests a personalized prognostic profile, identifying a patient to be profiled, establishing the user's identity and right to access the profile, selecting a number of user-specified options for the creation of the profile, and, in some cases, entering, uploading, or indicating the location in a specified database of new or additional clinical data about the patient of interest.

In some example embodiments, users may access the personalized prognostic profile management system 101 by interfacing via a webpage, application, app, or the like. That is, for example, each user is associated with credentials (e.g., user name and password pair) managed by the personalized prognostic profile management system 101. A user may attempt to access the personalized prognostic profile management system 101 by utilizing his or her client computing device to access a web page managed and/or provided by the report management system and, inputting his or her credentials (e.g., user name, password, biometrics). If the credentials are verified (e.g., by the report management system 101), the user is granted access. In some example embodiments, each user is allowed access to specified personalized prognostic profiles and/or data associated with specific patients, based on access control rights managed by a system administrator and implemented by the personalized prognostic profile management system. A user may view and/or edit a personalized prognostic profile based on the type of rights and/or level of access given to that user in connection with the patient who is the subject of the profile. For example, a physician might have the right to request and view a personalized prognostic profile on any one of a panel of patients under her care, while a patient may have access only to his own profile, with several of the user-specified options pre-selected by his attending physician. The systems and methods may be delivered via a web service or via client-server architecture with a large private network. The system stores and access information on a large reference database and must perform complex statistical computations essentially in real time. The system may have thousands of simultaneous users, and it must securely store and manage protected health information on many (e.g., millions) of patients. Because of the extensive demographic and clinical detail that must be stored on the patients in the reference database and actively utilized in the creation of personalized prognostic profiles, the reference database cannot be de-identified. While of course the names, exact birth dates and other explicit identifiers of patients could be removed after all necessary matching of patients and outcomes was done, the rich detail necessary on each patient would enable the patient's identity to be deduced by a skilled person who was determined to do so. Protection of the personal health information requires its storage in encrypted form, adding an additional computational load in model building, as data must be decrypted in real time in the process of analyzing it. Thus, computational capacity of mainframe proportions—or its equivalent in the cloud—is relied upon by the systems and methods described herein.

### Process

FIG. 2 illustrates a flowchart 200 for providing personalized prognostic profiles, according to an exemplary embodiment. As shown in FIG. 2, at step 250, one or more reference databases are stored, for example, in a memory associated with a personalized prognostic profile management system. The reference database includes data on a plurality of patients, their demographic, clinical and contextual characteristics, and their outcome(s) of interest over the system time frame as retrieved from various databases (e.g., the National Death Master File, structured data from specific care settings, databases of physiologic measurements transmitted from wearable devices), (e.g., FIG. 1, databases 109) and/or electronic health record systems (e.g., FIG. 1, health record systems 111). In some example embodiments, the patient data includes information associated with a deceased patient and/or the circumstances of the deceased patient's death. For example, the patient mortality data may include, for each patient in the mortality database, demographic factors (e.g., name, date of birth, age, date of report), diagnoses (e.g., date of assessment, assessment type, primary physician), functional status, symptoms (e.g., pain, anxiety, nausea, depression, confusion, lethargy, dyspnea), treatments, and the like. In some example embodiments, the demographic factors, diagnoses, functional status, symptoms, treatments, and the like, may be referred to as predictor variables. The reference database is described in further detail below.

In turn, at step 252, data (e.g., patient data) is received, for example, from a client computing device (e.g., FIG. 1, client computing devices 105). In some example embodiments, the received patient data is associated with an index patient. The patient data received at step 252 may include predictor variables, such as those described above with reference to step 250. The received patient data (e.g., index patient data) may be stored in a memory associated with a report management system, at step 254.

In turn, at step 256, a personalized prognostic profile corresponding to the index patient is generated. In some example embodiments, the personalized mortality prognostic report is made up of one or more widgets, interfaces, sections or the like, such as a personalized survival graph and an end-of-file management widget. Personalized survival graphs and end-of-life issues management widgets are described in further detail below with reference to FIGS. 3A-3C.

In turn, at step 258, the personalized prognostic profile is caused to be displayed at a client computing device. For example, a report management system (e.g., FIG. 1, management system 101) may transmit the personalized prognostic profile to a requesting user via the user's corresponding client computing device. In some example embodiments, the personalized prognostic profile management system is displayed at the client computing device via the application, web page, or the like managed and/or provided by the report management system.

FIG. 3A illustrates a personalized prognostic profile 300A, according to an exemplary embodiment. The personalized prognostic profile includes a personalized survival graph 301A, which may also be referred to as a personalized prognostic graph (e.g., in a more general embodiment). In some example embodiments, the personalized survival graph is the heart and/or central component of the personalized prognostic profile. The personalized prognostic profile may include other components (referred to herein as “widgets”) (e.g., 303A, 305A, 307A, and 309A), which are displayed next to and/or near the personalized prognostic graph or are conveniently linked to it. That is, for example, when the personalized prognostic graph is displayed (e.g., via a web page), the other widgets are either on the same page as the graph, on a page or pages linked to the graph, and/or on one or more popups linked to the graph. In another example, when the personalized prognostic graph is printed, the other widgets are printed on the same side of the same page, on the reverse of the same page, or on an additional page or pages physically attached to the page with the graph, or delivered along with it, e.g., in a common envelope. The other widgets may be used, for example to:
Identify the patient (e.g., 303A);
Identify the user and category of user (e.g., physician, family caregiver);
Indicate the requirements for the matched population and for report content that were specified by the user when making the request for the profile;
Explain that the personalized prognostic graph shows the actual experience of a matched population;
Explain how the matched population was derived (e.g., 307A);
Provide details of the predictive models used to create the matched population, including measures of model accuracy and identification of the most important predictor variables in the models; and
Give one or more checklists of action steps or considerations for the patient or other user, the necessity and/or timing of the steps being related to the patient's prognosis (e.g., 305A (symptoms and treatments checklist) 309A (e.g., end-of-life issues checklist)).

In some example embodiments, the widget for identifying a patient (e.g., displaying patient information) (e.g., widget 303A) is a required widget of the personalized prognostic profile. In some example embodiments, inclusion and/or display of other widgets (e.g., widgets 305A, 307A and 309A) depends on the context in which the personalized prognostic profile is being used. For example, if the personalized prognostic profile is used in the context of end-of-life care planning, the personalized prognostic graph is a personalized survival graph, and widgets may include: text that explains that the graph shows the actual mortality of patients very similar to the index patient, checklists that deal with palliation of symptoms such as pain or shortness of breath; physician orders for life-sustaining treatment; and legal issues such as a healthcare proxy or a durable power of attorney.

In some example embodiments, the widget 305A may be elaborated and/or generated as desired by a user, and output in real time to the patient's electronic medical record. If the user is a patient and the profile is presented as an active web page rather than a printed report, the widget 305A could be used to, among other things, prompt the patient's notification of the physician that a symptom was worse or was not adequately controlled.

In some example embodiments, the widget 309A includes physician orders for life-sustaining treatment (POLST). Each column in the checklist of widget 309A may be filled in manually or be filled in automatically via uploaded data from electronic medical records, subject to point-of-service modification by the user. User input may be automatically imported into the electronic medical record is the system is so configured.

In some example embodiments, the details about the matched population provided in the widget 307A can be greater or lesser than what is displayed in FIG. 3A, according to the context and needs of the user. For example, for certain patient and family users, it might suffice to show the number of matched patients and the exact matches required. Some professional users might want to see not only the top risk factors, the important time intervals and the earliest starting point for the matched population, but also statistics for model accuracy, such as the receiver operating characteristics or the predictive powers for the models for mortality (more generally, occurrence of the outcome) during the first and second specified time intervals of interest. These requirements for details of the matched population and the underlying predictive models are inputted by the user or may be determined by default based on the type of user, according to rules specified by a system administrator.

In some example embodiments, the widgets are personalized, both as to what topics they cover and the language in which the graph and the population matching process are explained. When the user is a patient with mild cognitive impairment or limited education, the explanation of the personalized prognostic graph is written with a simple vocabulary and with simple sentence structure. When the user of the personalized prognostic profile is a physician or a lawyer, explanations and checklists may use appropriate technical terms.

The common elements of the personalized prognostic profile are similar enough in design that the personalized prognostic profile may be used as a common language for discussions of prognoses between various stakeholders in the healthcare process. In this way, personalized prognostic profiles can have a major role in informing physicians and other healthcare professionals, patients and families, and other stakeholders in discussions of mortality prognosis and advance care planning for the end of life.

In some example embodiments, further explanation of the personalized prognostic profile, the personalized prognostic graph and the various widgets, tailored to the background and experience of a user, may be provided via links to educational materials. These materials may comprise without limitation text, diagrams, audio recordings, video recordings and/or interactive learning modules that may have adaptive features.

The systems and methods described herein are applicable to any binary outcome of interest that might or might not occur over a time interval of interest—whether a clinical one or a non-medical one. Likewise, the reference database of potentially predictive variables may include, in addition to demographic factors (which are typically included because of their usefulness to personalization), variables related to the geographical location, physical and social environment of the person of interest, the setting or system of care, and/or variables related to the person's educational, legal, and financial history, the person's interaction with social media and with channels of communication, data from self-rated and caregiver-rated questionnaires, and data transmitted from mobile devices

Predictor variables for medical outcomes (in addition to demographic variables that typically are included) may include following:
Standardized datasets from specific healthcare settings such as the Minimum Data Set (MDS) for skilled nursing facilities or the Outcome and Assessment Information Set (OASIS) for home health care;
Results of functional assessments;
Variables extracted from electronic medical records;
Formal codes for diagnoses and procedures;
Medications given;
Supplements and over-the-counter medications taken;
Data from surgical and other procedure notes;
Laboratory test values;
Results of imaging studies and other diagnostic tests such as electrophysiological tests;
Results of biopsies;
Data concerning the patient's genome, proteome, or metabolome;
Data concerning the patient's microbiome;
Physiologic data from clinical settings such as vital signs;
Physiologic data transmitted from or recorded by mobile devices or monitoring devices in non-clinical settings;
Symptoms reported by patients or caregivers on questionnaires or through interaction with devices;
Data from rating scales or screening tests, whether completed by a patient, a caregiver, or a health professional;
Variables based on records of financial transactions or legal actions;
Variables describing the process or content of electronic communications or interactions with social media;
Geographical and environmental data, including data on climate and microclimate, environmental toxins and infectious agents and allergens; and
Variables describing the physical setting of care and the auspices of care (e.g., Managed Medicaid plan, Veterans Administration health system, regional integrated care system, unmanaged private practice).

Examples of binary outcomes for which personalized prognoses can be developed utilizing the systems and methods described herein may include:

Clinical:
Recurrence of a malignant tumor;
Return to work following a work-related illness or injury;
Rejection of a transplanted organ;
Loss of vision to the point of legal blindness;
Remission of major depression (the binary outcome is the drop in a depression rating scale score below a specific point);
Virologic remission from hepatitis C infection;
Drop in CD4 count below the threshold for diagnosis of AIDS; and
Relapse of an addict to substance use after a period of abstinence.

Social:
Arrest for a criminal act following release from prison;
Default on a loan payment;
Completion of a month of full-time work after a period of unemployment; and
Episode of domestic violence after a period without violence.

One aspect of the personalized prognostic graph is that, in addition to providing the probability of a specific event (e.g., death or survival) occurring with a specific time frame, the graph provides and/or displays a pattern of risk over time. This pattern of risk over time advantageously communicates a picture of the future that optimally informs decision making.

For example, assume that groups of patients each have 50% chance of survival at six months. Group A has only 60% survival after 30 days, but ⅚ of the patients who survive 30 days will survive six months. Group B has 90% survival at 5 months, but 4/9 of those alive at 5 months will die in the following 30 days. End of life care planning has greater urgency for a patient whose matched population is Group A than one whose matched population is Group B. Now suppose that the 6 month survival of Group A is 55% and the 6 month survival of group B is 40%, but that their 30-day survival expectations are 60% and 90% respectively, as before. If choosing a specific treatment puts a patient in Group A instead of Group B, that treatment might be preferred by a patient willing to accept a 40% risk of dying within 30 days to get a greater likelihood of living longer afterwards, but rejected by a patient for whom that short term risk would be unacceptable.

### Selection of a Reference Population and Creation of a Reference Database

The systems and methods described herein may be used to create personal prognostic profiles for a given outcome, over a given time frame. The system time frame must be at least as long as any personal time frame a user will be allowed to request. The variables available on each person in the reference population, in some example embodiments, include—either directly or via acceptably accurate imputation—the variables likely to be available to describe the index patient, whether submitted by the user or obtained and/or derived from the same sources as the potential predictor variables in the reference database. The reference population typically consists of more than 100,000 patients (more generally, individuals at risk for the outcome of interest) and, in some embodiments, may consist of more than 1,000,000 patients, so that the matched population will typically consist of more than 100 patients and, in some embodiments, more than 1000 patients.

### User Inputs

Users of the system are properly authorized to see protected health information regarding the patient. Confirmation of this authorization may be either intrinsic to or extrinsic to the system.

The user of the system provides inputs via a user input interface or personalized prognostic profile request page (e.g., FIG. 3B, 300B). The personalized prognostic profile request page may be delivered via a web service as a web page. Using the personalized prognostic profile request page 300B, a user may provide user inputs such as:
The identity of the user (e.g., patient, physician, family member: this may determine what options the user will have in specifying the report, what widgets may be displayed, and the language and vocabulary used within the widgets);
The patient, identified sufficiently to access patient-specific data that are stored in the system, stored externally, uploaded and/or manually entered immediately prior to the generation of the personalized prognostic profile;
The personal starting point;
The personal time frame;
The forced match variables;
One or more time intervals after the personal starting point, no such interval being longer than the personal time frame. If there is more than interval selected by the user they are ordered by their importance to the user. The user-specified time intervals are ones of special importance to the user considering the patient's prognosis (e.g., 6 months because life expectancy of 6 months or less is a criterion for Medicare hospice eligibility, or 30 days because the patient hopes to celebrate a major life event 30 days after the personal starting point and is considering a treatment that might increase 30 day mortality risk as the price of potentially longer survival afterward);
One or more treatments that the patient might or might not receive (or begin to receive) within a specified number of days after the starting point. In the case that no treatment is under consideration, at least two different time intervals after the starting point are specified;
The earliest starting date that will be permitted in the subset of the reference population that will be used to create the matched population. This enables the user to decide how recent the data on other patients must be to be relevant in his or her view to the prediction of the index patient's likely outcome; and
Which widgets related to the patient's prognosis, care, treatment, and decision-making will be included in the personalized prognostic profile.

Each of the user inputs in the personalized prognostic profile request page may be divided into categories, as shown in FIG. 3B, including: user data, required exact matches, prognostic matches, technical details requested, and/or widgets requested.

When the personalized prognostic profile is delivered to the user via a web service, the request page typically will be a web page as well—as a form for entering information. Explanation of the personalized prognostic profile tailored to the background and experience of the user may be included within the widgets or may be provided via links to educational materials or by physical association with a printed profile. These materials may comprise text, diagrams, audio recordings, and/or video recordings.

Exact match requirements can be presented as multiple choice via pull-down menus or check boxes. The order of required exact matches may vary. The system will push a message to the user if the combination of required exact matches, important time intervals, treatment consideration and earliest starting point for the matched population cause the matched population to be too small. The user can then eliminate requirements until the matched population is sufficiently large. The system has default values for all matching requirements, whenever possible these will make use of attributes of the patient drawn from the patient's electronic medical record—less experienced users usually will accept them rather than override them with personal preferences.

Matching is done on estimated prognosis for the important time intervals, in order. The final step is contrasting treatment with no treatment.

A professional or technical user can request the “behind the scenes” description of the nested predictive models used to produce the matched population.

Many widgets can be offered, tailored to the needs of particular types of end users of the Profile. The report requester (the “user” of the system for producing the report) can specify which widgets are relevant in the current context. Additional widgets can be added over time, as the end user considers the information in the profile.

When the Profile is used to assess a treatment under consideration (in comparison to no treatment or usual treatment) a non-physician user usually will benefit from seeing a description of the treatment, its potential benefits, and its typical risks and adverse effects. It can be especially valuable to present this contemporaneously (and on the same page) with the historical experience of matched patients who did and did not get the treatment.

For each one of the user-specified criteria for the personalized prognostic profile (apart from the identification of the index patient and the user) pre-specified default values are stored and/or provided by the system; these may be based on characteristics of the user and/or the patient, according to predetermined rules set by the system administrator. For example, in the case of death or survival as the outcome, the default values of the personal time frame and the intervals of interest might be six months and 30 days. The exact matching requirements may be presented as multiple choice via, for example, pull-down menus or check boxes. The matches may be provided in any desired order. If the combination of required exact matches, important time intervals, treatment consideration and earliest starting point for the matched population causes the matched population to be too small, the system provides (i.e., pushes) a message to the user with this information. In response, the user can eliminate requirements until the matched population is sufficiently large. The system has default values for the matching requirements and, in some example embodiments, the default values are attributes of the patient drawn from the patient's electronic medical record selected according to predetermined and stored rules. In one exemplary embodiment, in the absence of user specification of matching requirements, age (as a range) and gender and primary diagnosis would be forced matches, and race would be added as an additional forced match when the medical literature supports the hypothesis of a different prognosis of the primary diagnosis or different response to treatment in patients with a specific racial background.

In some example embodiments, professional or technical users may request “behind the scenes” details and/or description of the nested predictive models used to produce the matched population. Examples of these details include the mathematical form of the predictive models, measure of model accuracy, and the most important predictive variables driving the models' estimates of the patient's prognosis.

In some example embodiments, a user requesting a report may specify which widgets are relevant in a particular context or use for the prognostic profile. Additional widgets can be requested and added over time, as the user considers the information in the profile. Widgets may in some embodiments be interactive web pages that support an adaptive learning process. Default combinations of widgets may be added based on the identity of the user that will be included along with the personalized prognostic graph unless explicitly excluded by a user request.

### Creation of a Matched Population

The personalized prognostic graph displays, to a user of the system, the actual outcomes of a matched population, with the intention that the user will view these outcomes a valid and credible predictor of the index patient's likely outcome over the personal time frame. This requires a matched population of adequate size (e.g., at least 500 patients, more than 1000, no fewer than 100; adequacy of a matched population will be related to the display interval, the index patient's risk of having the outcome of interest with the interval, and the purposes and requirements of the user), comprising patients that exactly match the index patient on all of the forced match variables, and meet other requirements that make their outcomes pertinent to predicting index patient's outcome, as explained below in further detail.

The matched population (referred to herein as “P_{M}”) is created from a subset P₀ of the reference population that matches the index patient on all of the forced match variables. In the case of age as a forced match variable, the match is performed on an age range. In the case of race as a forced match variable, the match may be narrower (e.g., Chinese) or wider (e.g., non-white) than a conventional racial category (e.g., Asian), with the matching criterion considering the representation of patients of different racial backgrounds in the reference database, and also whether a particular racial subcategory is known to be relevant to the patient's likelihood of having the outcome of interest. The database of outcomes and potential predictor variables for the population P₀ includes one instance of each discrete patient—typically one with the most recent starting point and/or the most complete set of potential predictors. The subset of the reference population with all of the required matches is referred to as P₀. To get from P₀ to P_{M} several predictive models—in all embodiments no fewer than two—are created and applied as follows:

Initially, the database relating to the population P₀, that is used to generate a predictive model, is created for the occurrence of the outcome of interest over the first of the important time intervals (i.e., T₁) specified by the user. This predictive model may be referred to as M₁. The predictive model M₁ may take any accepted form for the prediction of a binary outcome, including without limitation a logistic regression, a polynomial regression, a spline regression, a decision tree, a boosted regression, a lasso regression, a boosted tree, a random forest, a neural net or a support vector machine. The model is generated using an automated procedure. For example, a logistic regression may be created using a stepwise regression process based on all potentially predictive variables present in the reference database, or on a subset of potentially predictive variables that includes representation of several different categories of variables, e.g., demographics, diagnoses, symptoms, functional status, nutritional status, and vital signs. The automated procedure in some preferred embodiments has both a training component and a validation component. A randomly selected sample of 50% or more of the population P₀ is used to estimate model coefficients or train a neural net or support vector machine and the remainder of the population P₀ is used to validate the model estimated on the training data. If the model fails a validity test (e.g., does not reach a threshold of predictive power and accuracy) the system will then create a model with a different form. For example, if a logistic regression failed a validation test, a decision tree might be tried next. In one preferred embodiment, the system has sufficient computing power to develop and validity-test outcome predictive models in each of several different model forms, and then select the one with the best predictive performance on the validation sample. Alternatively, the bootstrap method may be employed to estimate the stability of each predictive model created by the system, and the stability of the model is considered along with its predictive power in the decision rule for selecting a model M₁. With some model forms a validation process is intrinsic to the construction of the model; when these forms are utilized other measures of model performance are used in comparing alternative models rather than the model performance on a discrete validation sample. Model M₁ is then applied to the index patient, resulting in an estimate R₁ of the probability that the index patient will have the outcome of interest within the first specified time interval T₁ after the index patient's starting point. A subset of the population P₀ is now selected comprising exactly those individuals with a predicted probability using Model M₁ that lies within a pre-specified interval around of the probability R₁, e.g., between 0.95×P₀ and 1.05×P₀. Call this reduced population P₁. The band around R₁ that is used to select P₁ is determined by the system by applying rules specified by the system administrator; in preferred embodiments the band will be narrower when the population P₀ is relatively large. The band may be described in terms of percentages above or below R₁, in terms of absolute differences in probability above and below R₁ or in terms of a requirement for a given number of patients with estimated survival probabilities that are closer than those for all other patients in the population P₀. The predictive model generation and subset selection process described above is iterated using the next specified important time interval (i.e., T₂). A predictive model M₂, which can take any accepted form for the prediction of a binary outcome and which is estimated on the population P₁, is created by an automated procedure, typically the same procedure used to create model M₁. M₂ estimates the probability that the outcome of interest will take place within the time interval T₂ after a patient's starting point. The model M₂ is applied to the index patient to estimate the probability R₂ that the outcome of interest will occur to the index patient within the time interval T₂ following the index patient's personal starting point.

In turn, a subset of the population P₁ is selected, consisting of those patients with a calculated probability, using model M₂ that lies within a specified percentage and/or predetermined range of the probability R₂. This selected subset of the population P₁ may be referred to as population P₂. If the reference population is sufficiently large and the user specifies additional important time intervals (e.g. T₃, T₄) the predictive model generation and subset selection process can be further iterated to eventually produce a population P_{M} that has been approximately matched on the predicted probability of the occurrence of the outcome of interest, at the conclusion of a series of successively specified time intervals Tᵢ. The end product of the process is thus the result of applying nested predictive models, such the predictive model at each stage is estimated on the population that was the output of the previous stage, and used to determine a subset of that population with a predicted outcome similar to that of the index patient.

Alternatively, if at any given stage in the process described the population Pᵢ is sufficiently large, the population may be divided by a random selection process into two mutually exclusive and complementary subsets Pᵢ* and Pᵢ**. The predictive model at that stage is created and validated on the subset Pᵢ* and then applied to the subset Pᵢ**. Members of the subset Pᵢ** with estimated probabilities of the outcome that lie sufficiently close to the estimated probability Rᵢ for the index patient are selected to constitute the population Pᵢ₊₁. This approach of “offset nested models” is employed where feasible to reduce the risk that the outcome predictive model at a given stage over-fitted—i.e., applicable to the specific population Pᵢ but not generalizable to other patient populations.

FIG. 3C illustrates a personalized prognostic profile 300C including a graph illustrating a comparison of survival of patients having versus not having received a specified treatment (e.g., treated vs. untreated patients), according to an exemplary embodiment. That is, in some example embodiments, the nested modeling process is carried out, if the user chooses (e.g., via a user input), to compare outcomes for members of a matched population who received a user-specified treatment of interest and those that did not. In such an embodiment, a predictive model is created on the population P_{M} that is the end product of the above-described nested modeling process applied to all of the user-specified important time intervals—i.e., the matched population. This predictive model crated on the population P_{M} estimates the probability that a patient will receive the user-specified treatment (i.e., treatment X). The model, M_{X}, is used to estimate the probability R_{X} that the index patient will receive the treatment X. In turn, a subset P_{MX} is selected from the population P_{M} that consists of exactly those patients whose probability of receiving treatment X lies within a specified interval around R_{X}. In some example embodiments the population P_{MX} consists of a specified number of patients that are closest to R_{X} in their estimated likelihood of receiving the treatment X, according to model M_{X}.

Alternatively, when the size of the population P_{M} is sufficient, the population P_{M} is subdivided by a random process into mutually exclusive and complementary subsets P_{M}* and P_{M}**; P_{M}*is used to estimate and validate the propensity model for treatment X, which is then applied to the patients in P_{M}**. The final matched population is a subset of P_{M}** consisting of exactly those patients with a propensity to receive the treatment X that lies within a specified interval of the propensity calculated for the index patient. This procedure of “offset nested models” is used where feasible to reduce the probability that the propensity model M_{X} will be over-fitted to the specific population P_{M} and not be generalizable to other patient populations.

More specifically, as shown in FIG. 3C, the profile 300C illustrates a graph 301C charting the percentage of surviving patients, among the matched population, having received a treatment (e.g., radiation therapy) versus those that did not receive a treatment, at user-specified time points (e.g., 0 months, 1 month, . . . , 9 months) measured from a starting point. FIG. 3C also includes two widgets 303C and 305C. Widget 303C includes a non-statistician's explanation of propensity matching and why it was performed. Widget 305C illustrates the matching process used to arrive at the charted survival figures of graph 301C.

In some embodiments the criterion for selecting the matched population P_{M} may include a requirement that for one or more of the time intervals Tᵢ the patients in P_{M} have risks of the outcome of interest for Tᵢ that lie in a proper subset of the interval around Rᵢ that was used for creating model Mᵢ. This may be done when the interval used for selecting Pᵢ from Pᵢ₋₁ was relatively large because a broader interval was necessary to ensure the stability of model Mᵢ. For example, the creation of model M₂ might be done on a population with risk at T₁ between 75% and 125% of the risk R₁ of the index patient, but the final matched population might require that patients' risk at T₁ lie between 90% and 110% of R₁.

In some example embodiments in which no potential treatment is specified and/or input by the user, the personalized prognostic graph shows and/or provides the actual outcomes of the population P_{M} over the personal time frame. In the case where a potential treatment is specified by the user, the personalized prognostic graph shows two outputs: (1) one showing the actual outcomes of those patients in the population P_{MX} who did in fact receive the treatment, and (2) one showing the actual outcomes of those in the population P_{MX} who did not.

The final output of the system is the personalized prognostic graph plus a set of additional widgets linked physically or electronically to the personalized prognostic graph. As described above, the additional widgets are used to, for example, identify the patient, the user, and the user's specifications for the personalized prognostic profile, such as their forced matches and personally important time intervals. The additional widgets related to items such as symptom control and advance care plans may consist of, for example, checklists to be filled in manually, or may include items with content that is pre-filled based on the patient's medical records or other relevant data sources.

In one example embodiment, a widget, which may be displayed by default, is and/or provides a listing of the prognostic variables that had the greatest weight in the predictive models of survival (more generally, predictive models of the outcome of interest).

The information presented to the user in and/or via the personalized prognostic graph comprises actual outcomes of patients, rather than model-based probability estimates alone. This makes the provided information more concrete and more accessible to a less numerate user. In addition, it makes evident that past experience is being used to make inferences about the future. The user-specified time limits on the past patient experience summarized by the personalized prognostic graph ensures that the data are generated at a time when medical practice and expected clinical outcomes are likely to resemble the practice and outcomes that will occur in the patient's immediate future.

Moreover, the information presented to the user in and/or via the personalized prognostic graph is based on the experiences of patients that are the same as the index patient on demographic, clinical, and contextual factors important to the user. This may be of great significance to the patient—e.g., the outcome of an 80-year old male veteran with lung cancer and posttraumatic stress disorder treated in the Veterans Administration health system may differ from that of a 55-year old female non-veteran with lung cancer but without posttraumatic stress disorder treated at a rural community hospital, even though the two patients may have the same primary diagnosis. For some users, ensuring that the personalized prognostic graph is based on the outcomes of patients with the same race or ethnicity may be critical to their acceptance of the prognosis.

The matching of the population on the probability of the outcome of interest over time emphasizes specific time intervals of special importance to the user, which may be in particular cases different from conventional and/or arbitrary intervals (e.g., six months). For example, a user may want to know the chance she will survive to be present at important event (e.g., a grandchild's wedding, an event scheduled to occur three months from the personalized starting point).

The comparison of the outcomes of those who did and those who did not receive a potential treatment is a comparison between patients who had a similar probability of getting the treatment, whether or not they did get the treatment. This technique, well known to statisticians as propensity matching, helps eliminate confounding of treatment effects with the effects of clinical differences between patients affecting a physician's decision to offer the treatment and the patient's decision to accept it.

The predictive models used in the nested modeling process described above can take any acceptable form for the modeling of a binary outcome. This maximizes the amount of variability that the models can predict. At the same time, the reporting of the predictor variables with the greatest weight in the nested models makes the process more comprehensible than if a simple model form were utilized but the key predictors were not disclosed to the user.

The periodic refreshing of the reference database, and the specification (by the user or by default) of the dates of acceptable reference data for a personalized prognostic graph make the system responsive to ongoing changes in medical practice and its outcomes. A prognosis based on “fresh,” individualized predictive models contrasts with a prognosis based on published literature that reports on research done with data several years old at the time the prognosis for the index patient is made.

In the models created by the nested modeling process describe above, typically, most of the predictor variables ultimately used are, in effect, interaction terms that reflect the operation of potential predictors of the outcome of interest within a particular demographic, clinical, and health system context. In many conventional algorithms for creating predictive models for clinical outcomes, few if any such interaction terms are tested for predictive utility, let alone used in the final predictive model, and indeed when the reference database is large and rich in potential predictors it would not be feasible to test every potential interaction among potential predictors, and the statistical significance of the models obtained would be diminished by adjustment for multiple comparisons. Yet, it is the interaction of user-specified forced matches with patients' other characteristics that makes the personalized prognostic profile described herein relevant and credible to the user.

The personalized prognostic profile is generated on demand, making it ideal to inform decision-making by physicians, patients, families, and other users. The on-demand feature makes it responsive to recent changes in the patient's clinical status and to the patient's life events, and to addressing new questions, such as whether a particular treatment makes sense for a given patient at the present time.

Generating personalized prognostic profiles is achieved by using large and efficient databases can be managed and with which predictive models can be estimated, validated, and applied. Moreover, computation of the personalized prognostic profiles may be performed in the cloud, where additional computing capacity can be accessed on demand. Advances in natural language processing also have a role in making the system feasible, particularly when important potential predictors are not available in a standardized, structured dataset but must be extracted from clinical notes.

The systems and methods described herein provide more than typical capabilities of existing statistical packages for exploratory data analysis and predictive modeling. Such existing statistical packages are not designed to create outputs for direct use by a range of users with widely differing backgrounds and levels of education, literacy, and numeracy, and they do not allow for extensive input by non-technical users that shapes the analysis and helps determine the structure of the output. At the same time, the capabilities of off-the-shelf statistical packages to estimate and validate a single predictive model—a step that is carried out at least twice in creating the personalized prognostic graph—are what enable the embodiment of the invention described herein by one skilled in the art of predictive modeling and statistical analysis.

### EXAMPLE 1

A reference database includes 5 million patient assessments, representing 2 million discrete individual patients who have had structured clinical assessments, mortality status for at least 15 months after each assessment, and indicators of whether various treatments were administered to the patient, including indicators of the use of cancer chemotherapy. The user, in this case the patient herself, is interested in the 6 month and 30 day mortality risks (in that order of importance) for a white woman in her 70s with recurrent metastatic breast cancer who has a similar prognosis to hers, with or without the salvage chemotherapy that is currently being considered.

The subset (sub-population) P₀ of the reference population comprising white women in their 70s with recurrent metastatic breast cancer at the time of a structured clinical assessment is selected and is found to have 30,000 patients, each with one or more structured clinical assessments that could be used as a starting point assessment, each with information about whether chemotherapy was given subsequent to the starting point, and each with information about the outcome of mortality over the 15 months (the system time frame) following the starting point. A dataset is created that has one baseline assessment for each discrete patient, a variable indicating whether chemotherapy was given, and the mortality outcome for each of the patients in the population P₀, where each patient's starting point for measuring survival is the date of her starting point assessment. When there is more than one structured assessment of an individual patient in the population P₀, the one that is selected as a starting point assessment is the most recent complete assessment of the patient, excluding assessments that are preceded by the initiation of salvage chemotherapy. A predictive model is developed (i.e., estimated on a model development subset of P₀ and validated on the remainder of P₀) to estimate 6 month mortality risk in the population P₀. The model is applied to the index patient and the population P₀ is reduced to a population P₁ of 8000 patients with an estimated 6 month mortality risk between 90% and 110% of the estimated risk for the index patient.

This process is repeated, beginning with patient population P₁ and, modeling 30-day mortality, applying the model to the index patient to calculate her expected 30-day mortality, and then selecting the subset of P₁ that has an estimated 30-day mortality risk between 90% and 110% of that calculated for the index patient. This population P₂ in this example consists of 1500 patients. A predictive model is now developed on population P₂ to estimate the likelihood that a patient in that population would receive salvage chemotherapy. This model is applied to the index patient to determine her likelihood of receiving the treatment, and a subset of the population P₂ is selected consisting of patients whose estimated likelihood of receiving salvage chemotherapy is between 85% and 115% of the estimated likelihood for the index patient. This subset is P₃, in the present example consisting of 600 patients. Of these, 400 received the treatment and 200 did not. The observed outcomes of the two groups are displayed on a personalized survival graph.

One widget accompanying the graph explains to the user that these are the actual survival data for “patients like you”, white women in their 70s with recurrent metastatic breast cancer whose mortality prognosis overall is similar to hers, and whose likelihood of receiving salvage chemotherapy in the patient's current system of care is similar to hers. Another widget shows that the most important factors related to her prognosis are her age, her being functionally independent and her not having heart disease. Yet another widget explains the effect of the proposed chemotherapy on her symptoms, function, and quality of life, so that these can be considered along with the expected effect of the chemotherapy on her risk of dying in the next six months. In this example the graph makes clear that with chemotherapy the 30-day mortality risk is higher, and the 6-month mortality risk is lower, in comparison to care without chemotherapy.

### EXAMPLE 2

Another detailed example of the creation of a prognostic graph shows how a survival curve for a matched population based on a forced matching, multiple time points of interest and nested predictive models can lead to fundamentally different conclusions about a patient's prognosis than the application of generic, non-personalized predictive models, even allowing for matching of prognosis at multiple time points.

The patient described in the following example is a composite of several actual clinical patients, but the data analysis described is an actual data analysis performed using electronic medical record data from an actual patient and an actual reference population. The latter fact implies that the example is a demonstration of the feasibility of the technology with structured clinical data actually available in healthcare systems.

Patient X, an 88-year old man with congestive heart failure, is interested in doing advance care planning for the end of life. He is a long term resident of a skilled nursing facility. He is happy there, but would not want to prolong his life if doing so involved hospitalization and potentially painful and invasive procedures. His family is concerned that he might forgo treatments that could prolong his life because of an unrealistically negative and pessimistic view of his mortality prognosis. In this case the patient, his family, and his physician all seek a clear picture of his outlook for survival. His physician has entered his clinical data into published algorithms for predicting heart failure mortality, but the patient and his family do not find the results of such calculations to be meaningful to them—raising the concerns that the patients studied in academic centers and reported upon in published articles might be different in important ways from Patient X. Also, they are aware that standards of medical practice and options for treatment are continually evolving. With this in mind, the patient and family like the idea of seeing the actual mortality outcomes of recently treated patients with heart failure who, like Patient X, are:
Over 80,
Do not have diabetes (a common accompaniment of heart failure and one that increases mortality risk),
Long-stay residents of skilled nursing facilities, and
Treated within the last three years (and thereby reflecting the results of recently prevailing standards of practice and options for treatment).

To create a Personalized Prognostic Graph for Patient X, the user (in this case his physician) interacts with the system's web page to indicate her interest in the mortality of long-term nursing home residents over 80 with congestive heart failure, treated in the past three years, without diabetes. She selects two time periods of interest: Six months (first priority) and 30 days (second priority).

The system for generating the Personalized Prognostic Profile selects a sample S of data—in this case structured assessment data from skilled nursing facilities using the Minimum Data Set for nursing home resident assessment—comprising patients treated in the past three years, who have complete mortality data available for at least six months following their starting point assessment, have heart failure, don't have diabetes, and are over 80. A subset of MDS items associated with 6 month and/or 30 day mortality for residents with heart failure is selected by the automated statistical procedure known as lasso regression; these items are the candidate predictor variables. In fact, the MDS database available to the system with the appropriate treatment dates and adequate follow-up data has 38549 patients with congestive heart failure; of these, 16896 are over 80 and do not have diabetes.

A predictive model utilizing the candidate predictor variables is generated by the system. The model is applied to estimate the 6-month month mortality risk for the patient and for all of the patients in the sample S, leading to the selection of a subset S1 of the sample comprising patients with estimated mortality between 75% of the estimated mortality for patient X and 125% of the estimated mortality for Patient X.

The process is now repeated for the outcome of 30-day mortality, carried out only on the data from the 3992 patients in the subset S1; i.e., from a subset of patients who not only match Patient X on the selected diagnosis and demographic, but also on their approximate 6-month mortality risk. A personalized predictive model is generated by the system and applied to Patient X and also to all of the patients in S1. The estimated 6-month mortality risk using the first model and the estimated 30-day mortality risk using the second model are both considered in the selection of a subset S2 of S1 comprising just those patients in S1 for whom the estimated 6-month mortality lies between 90% and 110% of the predicted mortality for Patient X and the estimated 30-day mortality lies between 90% and 110% of the predicted mortality for Patient X. There are 254 patients in the subset S2.

S2 is the matched population for Patient X. Patient X's personalized survival graph shows the actual survival of the population S2 over a six month period. His Personalized Prognostic Profile comprises this graph together with end-of-life issues widgets, in his case including a symptom control widget that addresses his symptom of shortness of breath (as well as other symptoms such as pain, nausea, and anxiety), and an advance care planning widget that addresses his request for a Do Not Hospitalize order in his nursing home medical record, and his appointment of his daughter as his Health Care Proxy, and his wishes regarding resuscitation and the use of various life-sustaining treatments.

This example contrasts with the prior art of estimating and presenting a mortality prognosis, which might develop along any of the following lines:

Case 1: The physician makes a qualitative judgment based on his experience that someone like Patient X has even odds of surviving for six months. He tells the patient and family that Patient X has a life expectancy of around six months. The patient interprets this as a prediction that he will die in six months. The patient's son does not think that his mother's doctor is clairvoyant and questions this conclusion. The patient's daughter, an actuary, correctly understands the physician as meaning that approximately 50% of patients similar to her father will die within six months.

Case 2: The physician utilizes a published predictive model for estimating the risk of death for patients with heart failure. The model estimates that 40% of patients who have short list of clinical features identical to those of Patient X will die within six months. The patient asks how many of the patients used to create the model lived in a nursing home and how many of those were over 80. The physician does not have this information at hand; when she looks it up he finds that almost all of the patients used to create the model were either hospitalized or were outpatients living at home. The patient and family question the accuracy of the prognosis given these facts. The quantitative estimate is in one way more “objective” than the experience-based estimate in Case 1, but it is not necessarily more accurate, nor does Patient X find it more credible than his physician's experience-based subjective opinion.

Case 3: The physician finds a predictive model for six-month mortality specifically created for patients with heart failure who reside in skilled nursing facilities. Applying the model to Patient X's data she gets 45.7% as the estimated probability that the patient will die within six months. The use of a setting-specific model makes the prediction more credible to the patient and his family than the estimate in Case 2, and the family finds it reassuring that it jibes with the physician's more subjective estimate. The actuary daughter understands the meaning of the estimate, but the patient has a hard time visualizing what the future will hold for him over the next few months.

Case 4: The physician accesses a system that generates a six-month survival curve for heart failure patients in nursing homes with clinical features similar to those of Patient—the black line in FIG. 3D. This patient relates to the curve as showing the outcome of “patients like me”, and gets the idea of a gradually accumulating risk of death over the next several months. This is more useful to him than the numerical estimate in Case 3.

Case 5: An improved system that selects patients for comparison using prognoses from more than one time point is applied to create a six month survival graph, this time comprising patients with estimated 6-month mortality risk and estimated 30-day mortality risk close to that estimated for Patient X, with the 6-month mortality risk estimate as in Case 4 and the 30-day mortality risk estimate created by a general model for 30-day mortality for heart failure patients in nursing homes. The latter risk is 4.1%; patients are selected for the graph is their 6-month mortality risk is between 43.4% and 48.0% and their 30-day mortality risk is between 3.9% and 4.3%. This produces a survival graph representing the actual outcomes of 110 patients, shown by the red line in FIG. 3D. The 6-month mortality rate in this group of patients is approximately 43.6%—lower than the estimate produced by the generic 6-month mortality model, and also that the graph makes clear that the mortality risk over the next month is relatively low at about 4%. Based on this more refined and comprehensible information the patient decides to take her time in making weighty decisions about forgoing treatments, seeking more opinions and information, because she now sees she is very likely to survive in the short term, and that her 6-month outlook is a bit better than she would have expected from her physician's initial subjective prognosis. The system's survival curve, while not yet personalized, is superior in accuracy and utility to the curve of Case 4.

Case 6: The system of the present invention is applied to create a predictive model for 6-month survival of nursing home residents with heart failure who are over 80 and don't have diabetes, based on data from patients treated in the past three years. The model is created and applied to the clinical data from Patient X, producing an estimated 6-month mortality of 47%, not significantly different from a generic model for heart failure mortality in nursing home residents that did not force matches on the patient's age and absence of diabetes—i.e., the model of Cases 3 and 4. However, though the estimate isn't significantly different from that produced by the generic 6-month mortality model, the patient and family find it more credible because the patients used to develop the model are more like Patient X, and because the data are from patients treated relatively recently. The survival graph—the green line in FIG. 3D—showing actual outcomes of patients with similar estimated mortality using the “forced match model” is likewise more credible to the patient and the family, with the forced matching bolstering the case that the graph shows the actual outcomes of “patients like you”.

Case 7: The system of the present invention creates a predictive model for 30-day mortality that starts with the forced match data (from long-term nursing home residents with heart failure who are over 80 and do not have diabetes), further limited to the 3992 patients whose estimated 6-month mortality by the model of Case 6 lies between 39% and 65% (that is, between 75% and 125% of the 6-month mortality estimate for Patient X under the forced match model). This “nested model” for 30-day mortality effectively is based on the interactions of potential predictor variables with the forced match variables and with the constellation of factors that determine the 6-month mortality prognosis. Using this model the 30-day mortality estimate for Patient X is 3.7%, 10% lower than the estimate from applying a generic model for nursing home residents with heart failure. The system then produces the survival curve for a group of nursing home residents with heart failure who are over 80 and don't have diabetes, who are close to Patient X on both their 6-month mortality estimate and their 30-day mortality estimate, the former calculated using the forced match model and the latter calculated using the nested model. In this case, “close to Patient X” means a 6-month mortality estimate between 90% and 110% of the 6-month mortality estimate for Patient X under the forced match model (in this case between 42.3% and 51.7%) and a 30-day mortality estimate between 90% and 110% of the 30-day mortality estimate for Patient X under the nested model (in this case between 3.3% and 4.1%). The result is the blue line in FIG. 3D, which shows the actual outcomes of 254 patients. The 6-month mortality in this group of patients is 24.4%—markedly lower than the mortality estimated by the physician subjectively, by the generic 6-month mortality model, by the combined 6-month and 30-day generic mortality models, or by the forced-match 6-month mortality model. The use of personalized modeling methodology that combines modeling on a forced matched sample, using multiple endpoints and nested models produces a prognosis that is more valid and more credible to the patient and family than a conventional approach to prognosis, whether that approach was purely subjective or one that made use of conventional, published predictive models. In this case, the mortality prognosis estimated by the personalized prognostic system is dramatically better than the prognoses generated by other methods, to a degree likely to influence the decisions of the patient and his family. The more valid, credible, and comprehensible prognosis shown by the personalized prognostic graph of the system of the present invention is a sound basis for shared decision-making by the patient and his physician. Patient X, viewing the personalized survival graph shown by the blue line on Figure Y might opt for more aggressive life-prolonging therapy than one viewing the non-personalized, conventionally-produced survival graph indicated by the black line or the red line.

Case 8: The personalized survival graph of Case 7 is surrounded by widgets dealing with end-of-life issues including symptom control, advance care planning, and legal and financial arrangements. The patient and family are prompted to address these important issues with a credible and comprehensible prognosis clear in their minds.

The models utilized in this example—both the generic ones and the personalized ones—were all generated by standard statistical software, in this case the “R” software package; all models utilized boosted regression methodology. Model performance for all of the models utilized is expressed as the area under the Receiver Operating Characteristic curve for the boosted tree predictive model. These model performance measures were 0.67 and 0.72 for the generic 6-month and 30-day mortality models, respectively, 0.66 and 0.80 for the forced match 6-month mortality model and the nested 30-day mortality model. The underlying data were from a database of skilled nursing facility Minimum Data Set assessments routinely maintained and clinically updated by PointRight Inc., a company providing web-based clinical analytics services to nursing facilities.

This example shows that the personalized prognostic graphs of the present invention can be generated from data it is practical to obtain, using standard statistical packages, and that the personalized models can have the same or greater predictive power as generic ones based on larger pools of clinical data. These model performance measures are competitive with measures for published models often used in clinical decision support. The description of the system herein enables the implementation of the system if sufficient clinical data are available and the system operator is skilled in the use of a statistical package like R or one of its competitors (e.g., SAS or SPSS).

Major electronic medical record companies, large-scale vendors of web-based healthcare analytics, large health plans, and analytics contractors for large health plans are four examples of entities that have sufficiently large and rich datasets to permit the implementation of the Personalized Prognostic Profile for a meaningful group of individuals. Individuals with rare conditions might best be served by the system described herein if the system could access clinical data from a healthcare system, payer or provider responsible for a large population suffering from their specific condition. For example, the datasets from cancer centers would be an obvious data source for generating Personalized Prognostic Profiles for various types of cancer patients.

Personalization of the Personalized Prognostic Graph

In some example embodiments, the degree of personalization possible using the systems and methods described herein depend in part on the size and representativeness of the reference database. If the reference database has little data about a particular demographic minority or about a particular medical diagnosis, forcing matches on these details will greatly decrease the size of the potential matched population. In some embodiments the system provide warnings to users that the system cannot accommodate all of their requested forced matches and still produce an adequate matched population to produce a credible personalized prognostic graph. The user will then have the opportunity to reduce the number of forced matches, concentrating on those most critical to the acceptance and use of the system's output.

In some example embodiments, if a patient has an especially high or especially low probability of receiving a treatment of interest, it will be necessary to accept fewer forced matches, different forced matches or a higher percentage of difference in estimated prognosis between the patient and the matched population in order to have an adequate sample size for a personalized prognostic profile. In some embodiments the system messages the user to this effect, so that the user can decide what compromises are acceptable to him or her in the creation of the matched population for the personalized prognostic graph. If personalized prognostic profiles emerge as a standard in healthcare, the need for a widely representative reference database will be understood in the healthcare industry, and various organizations are likely to create such databases whether for service to the public or for commercial reasons (or, likely, both).

Parameters to be Specified by the System Administrator

In addition to user-specified requirements and/or parameters for the personalized prognostic profile, several parameters needed for operation of the system may be specified by a system administrator rather than by the user, though the system may be configured to allow overriding of the default parameters by a qualified user. Users may receive notifications when the application of their forced matches combined with the pre-specified parameters produces a matched population of insufficient size. In some example embodiments the user has the option of modifying one or more of the system parameters, in addition to having the option of changing the forced match criteria.

That is, parameters that may be specified and/or input by the system administrator include, for example:
(1) The minimum size of the matched population. Typical values may range between 100 and 1000. Alternatively the minimum size can be determined by a rule or formula based on the personal time frame and the estimated risk of the index patient for the outcome of interest.
(2) The required closeness of the match with the index patient on the estimated probability of the outcome of interest over the most important user-specified time interval. A typical parameter might specify that if the index patient's estimated probability of the outcome is R, the estimated probability for the members of the matched population will lie between 90% and 110% of R; another typical parameter might specify that there will be 10,000 patients in the matched population, consisting exactly of those with estimated probabilities of the outcome of interest closer to that of the index patient than those not in that group of 10,000. Or, the system administrator might specify the interval in terms of differences in estimated probabilities above and below the estimate for the index patient, e.g., between 0.01 below to 0.02 above the estimated probability for the index patient. Narrower limits for selecting sub-populations at each step in the iterative process are feasible when reference populations are larger and forced matches are few and not unusual; broader limits may be desired when the reference population is small and/or the forced matches already limit the usable subset of the reference database to a small minority of the reference population.
(3) The required closeness of the match with the index patient on the estimated probability of the outcome of interest over the second most important user-specified time interval.
(4) Criteria for the required closeness of matches with the index patient on the estimated probability of the outcome of interest over user-specified time intervals of the third or lesser level of importance.
(5) The required closeness of the match with the index patient on the estimated likelihood of receiving (propensity to receive) the treatment of interest, if a treatment is being evaluated by the system.
(6) What widgets will be added by default to the personalized survival graph, based on attributes of the patient and the user (if the latter is different from the patient), as well as the user's selections of options, forced matches, and time intervals of interest.

### Pre-Calculating Matched Populations or Personalized Prognostic Graph Data

Each step in the process of selecting a matched population requires the construction and validation of a predictive model, based on a dataset of particular relevance to the patient of interest and his or her clinical situation. The actual model form can be, without limitation, a logistic regression, a polynomial regression, a spline regression, a decision tree, a neural net, a random forest, a boosted regression, a lasso regression, a boosted tree or a support vector machine. As new methodologies for predictive modeling of binary outcomes become available they can be applied. Software for predictive modeling can operate on subsets of the reference database to create and validate models of various types in real time, responsive to user selections of forced match variables and of time intervals and treatments of interest, user-required matches of risk factors, and the user's time points of interest.

In some example embodiments, the systems and methods described herein provide the ability to identify a number of common combinations of forced match criteria, time intervals of special importance and potential treatments of interest, and pre-calculate predictive models. This can accelerate the first step in the creation of the nested populations that culminate in the matched population. The initial predictive models for the outcome of interest in the population with all of the forced matches may be pre-calculated and stored, for many common combinations of forced match criteria. The subset P₁ that will be selected from the population P₀ will be different for different index patients, depending on their estimated probability of having the outcome of interest over the first important user-specified time interval and the pre-specified interval around the index patient's estimated risk that will be used to select the subset. Differences in the population P₁ may change the details of the predictive model for the outcome over the second user-specified time interval, or change the details of the predictive model for receipt of the treatment of interest.

### Continual or Periodic Updating of the Reference Database

Modeling the risk of the outcome of interest (e.g., mortality risk/survival probability) and selecting matched populations on demand allows models to be created and matched populations to be selected, automatically reflecting recent changes in standards of care and usual clinical outcomes. If for example a one-year interval is the longest one to be covered by the system, the dataset of patient assessments and mortality outcomes might comprise data in which the starting point assessments were done no more than three years prior to the creation of the personalized survival graph. In one example embodiment, the underlying reference population of patient assessments and associated mortality outcomes is updated quarterly.

Despite the continual updating of the reference database a consequential and rapidly-disseminated medical innovation—such as a new antibiotic efficacious for an otherwise life-threatening infection—or a recent epidemic condition—could for specific patient populations make the predictive models, matched populations and personalized prognostic graphs built by the system inappropriate as a basis for prediction of an index patient's outcome. To deal with this, the system may in preferred embodiments store such “exceptions” and push a notice to the user when the index patient's characteristics implied that the prognosis could be affected by very recent changes in practice or epidemiology. In most cases, however, a physician would be involved in the case and would communicate with the user about this limitation of the automated personalized prognosis.

### Reference Database

The reference database may include the following attributes:
(1) Data in the reference database may be structured from the outset or converted into structured data by natural language processing. The database comprises variables with values that are binary, categorical, or ordinal, or real numbers; vectors of such values; or other data types such as images or recordings of physiological data that the system converts into numerical or categorical values or vectors of such values. The variables are derived from a standardized dataset or are extracted from medical records or other data sources via analysis of free text (e.g., medical progress notes or procedure notes), by mapping items from another source to the database (e.g., porting laboratory values from the laboratory report section of the electronic medical record system to the reference database), and/or by automated image analysis or pattern recognition, as might be used in utilizing diagnostic imaging data or in creating variables from the data feed from a physiological monitoring device. Categorical data may be converted into binary or ordinal items to facilitate their use in predictive models based on calculations.
(2) Data in the reference database includes complete data on the outcome of interest. For example, in the case of death as the outcome, these data may be based on death certificates or on a state or national database of deaths such as the Social Security Death Master File. To be used in creating a personalized survival curve that runs for time T after the most recent clinical assessment (e.g., the system time frame=T), the mortality data for each patient in the reference database must be known for up to time T following the last assessment date. That time T might be one year or more. In such an exemplary case the starting point assessments in the reference database might have dates approximately two to three years prior to the last database update. That is, the dates would belong enough in the past for patients' outcomes to be completely known but recent enough so that the patients' outcomes would be relevant to the expected outcome for a patient assessed at the present time.
(3) A given reference database is created at a fixed point in time, and used to generate personalized survival graphs until it is updated with by data that are either more recent, more accurate, and/or more comprehensive. Ideally the reference database used for a patient assessed in a particular clinical setting—e.g., hospital, outpatient, or skilled nursing facility, would be based on data from and about patients assessed in the same clinical setting. However, this is not essential, as long as the site of the assessment were one of the variables in the database that could be used in matching and modeling.

Once the variables in a reference database are defined they are evaluated for statistically significant relationships with the outcome of interest within each of several specific time intervals Ti after the date of the assessment. These time intervals are the ones that users would be able to select as time intervals of interest to them when a personalized survival graph is created. Examples of these intervals are 30 days, 60 days, 90 days, 120 days, six months, nine months, and one year. Each variable will be tested in a simple univariate model (e.g., logistic regression or decision rule) for its relationship with death within each interval Ti. Variables associated with p values below a specified threshold, e.g., p<0.01 are designated as candidate predictor variables for the outcome of interest within the time interval Ti.

Once the initial set of candidate variables is determined, additional derivative variables are generated and tested for association with the outcome of interest within time interval Ti. These comprise without limitation principal components from a principal component analysis of the predictor variables, interactions of two or more predictor variables, and summary scales or counts of multiple predictor variables, including prognostic or condition severity indices drawn from the medical literature. Each of these derivative variables is tested for association with the outcome of interest within the interval, and it will be added to the candidate predictor variable list if it has a particularly high p value and/or in itself it explains a relatively high proportion of variance in the mortality outcome. Basic demographic variables such as age, sex, and race, and the principal diagnosis are included among the candidate predictor variables and are tested in interaction terms during the process of creating the final candidate predictor variable list. Age may be broken into ranges that then become binary variables. If the setting of care is not the same for all patients using the system, the setting of care is on the candidate predictor list, and various interactions of the setting of care with other candidate predictors are tested. At the conclusion of the process the list of candidate predictor variables may be reduced in number by applying rules specified by the system administrator. For example, the cutoff for p-values might be less than 0.01, or if a group of candidate predictors are highly correlated with one another a single example of the group would be retained for use in subsequent modeling procedures.

In some example embodiments, the personal prognostic profile may be embedded within electronic medical record/electronic health record so that it can be accessed at any time by a user of the record. Access to the electronic record might be via a mobile device. In some embodiments the mortality risk estimates (e.g., outcome probability estimates) are be updated automatically if new data on the index patient is entered into the patient's record that changes a value of one of the prognostic variables used in the predictive models underlying the selection of the matched population and calculation of the personalized prognostic graph. In these embodiments a message might be pushed to the patient's physician that the estimated prognosis has changed.

In some example embodiments, the systems and methods described herein may offer a “what if” mode in which a user can enter variables outside the patient's medical record and get an analysis of the survival or other outcomes of a matched population that is similar to the index patient with the “what if” conditions as specified. For example, the system might enable the user to visualize, for a patient with end-stage chronic lung disease, how the prognosis would change if the patient stopped smoking cigarettes, or, for a socially isolated patient living alone, how the prognosis would change if the patient lived in a group setting.

In some example embodiments, the personalized prognostic profile is specific to a given healthcare system, health plan, or a national health service. In such an embodiment, the reference database is sufficiently large and mortality data (e.g., data on the outcome of interest) are available. The personalized prognostic profile may be adapted to a national health system like the one in the UK or the one in Sweden, where there is already a great deal of standardized data collected and centrally stored.

### Widgets Used in the Mortality Prediction/End-of-Life Care Planning Embodiment

### Palliative Care Issues Widget

This widget includes a specialized listing of symptoms frequently encountered at the end of life. The symptoms include pain, shortness of breath (dyspnea), anxiety or fear, depression, sleep disturbance, nausea, and constipation. Other symptoms not listed here might also be included if relevant to the clinical condition of the patient. In some example embodiments, the listing of symptoms is in the form of a checklist, indicating, for each symptom, whether the symptom is present and whether the symptom is being addressed by a specific intervention. There may be in addition ratings of symptom severity on an ordinal scale (e.g., mild, moderate, severe, very severe; or 1-10). In some example embodiments, the widget includes an indication of whether treatment has been effective. This content may be filled in manually by a clinician, patient, caregiver or other user, or transferred from an electronic health record.

### Advance Care Planning Widget

This widget includes a listing of items related to advance care planning such as Do Not Hospitalize or Do Not Resuscitate orders, Physician Orders for Life Sustaining Treatment, preferences regarding organ donation, and utilization of palliative care services or election of a Medicare or commercial health plan's hospice benefit. With each item, the widget includes an indication of whether the patient (or the patient's proxy) has addressed a particular issue related to end-of-life care and/or advance directives. For some items, there may be a space for indicating that the specific issue is not applicable to the patient. For items addressed, there may be the option of noting the date when it was addressed (e.g., when a healthcare proxy was appointed) and for making comments, mentioning a name or providing contact information.

### Legal and Financial Issues Widget

This widget includes a listing of items related to the patient's legal and financial issues, such as having a durable power of attorney, directions for disposition of assets, and issues concerning trusts.

### Emotional and Spiritual Issues Widget

This widget includes a listing of items related to the patient's emotional and spiritual issues, including religious concerns, potential involvement of clergy, need for counseling or psychotherapy, and unfinished business within the family.

By virtue of the systems and methods described herein provides a number of valuable advantages. For example, the combination of forced matching and nested models gives meaningfully different results than using general-purpose mortality models or even models built on a forced matched population but without nesting and combination of models. Results can differ enough to lead physicians and patients to different conclusions regarding advance care planning and choice of treatments. The approach of forced matching and nested modeling has greater face validity than the application of a published predictive model developed on a generic population, and it is usually will have greater predictive validity as long as the size of the reference population is adequate to support the forced matches and other user preferences and still yield a matched population of sufficient size for stability of risk estimates. The practical significance of the difference is demonstrated by Example 2 and FIG. 3D above, that show in a particular case that the estimated 6 month mortality using a generic model is almost twice as high as the personalized 6-month mortality prognosis based on forced matching and nested models.

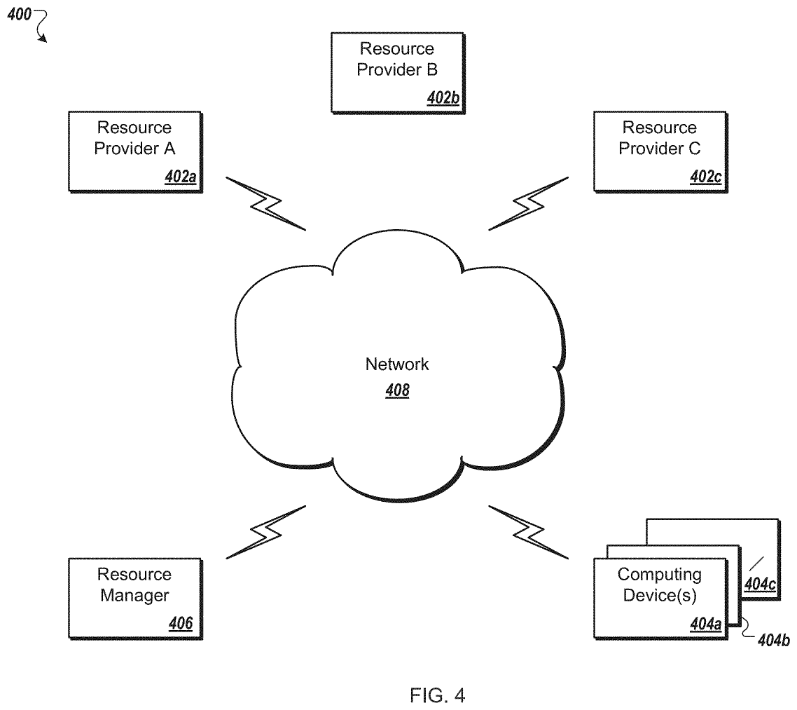

FIG. 4 shows an illustrative network environment 400 for use in the methods and systems described herein. In brief overview, referring now to FIG. 4, a block diagram of an exemplary cloud computing environment 400 is shown and described. The cloud computing environment 400 may include one or more resource providers 402a, 402b, 402c (collectively, 402). Each resource provider 402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 402 may be connected to any other resource provider 402 in the cloud computing environment 400. In some implementations, the resource providers 402 may be connected over a computer network 408. Each resource provider 402 may be connected to one or more computing device 404a, 404b, 404c (collectively, 404), over the computer network 408.

The cloud computing environment 400 may include a resource manager 406. The resource manager 406 may be connected to the resource providers 402 and the computing devices 404 over the computer network 408. In some implementations, the resource manager 406 may facilitate the provision of computing resources by one or more resource providers 402 to one or more computing devices 404. The resource manager 406 may receive a request for a computing resource from a particular computing device 404. The resource manager 406 may identify one or more resource providers 402 capable of providing the computing resource requested by the computing device 404. The resource manager 406 may select a resource provider 402 to provide the computing resource. The resource manager 406 may facilitate a connection between the resource provider 402 and a particular computing device 404. In some implementations, the resource manager 406 may establish a connection between a particular resource provider 402 and a particular computing device 404. In some implementations, the resource manager 406 may redirect a particular computing device 404 to a particular resource provider 402 with the requested computing resource.

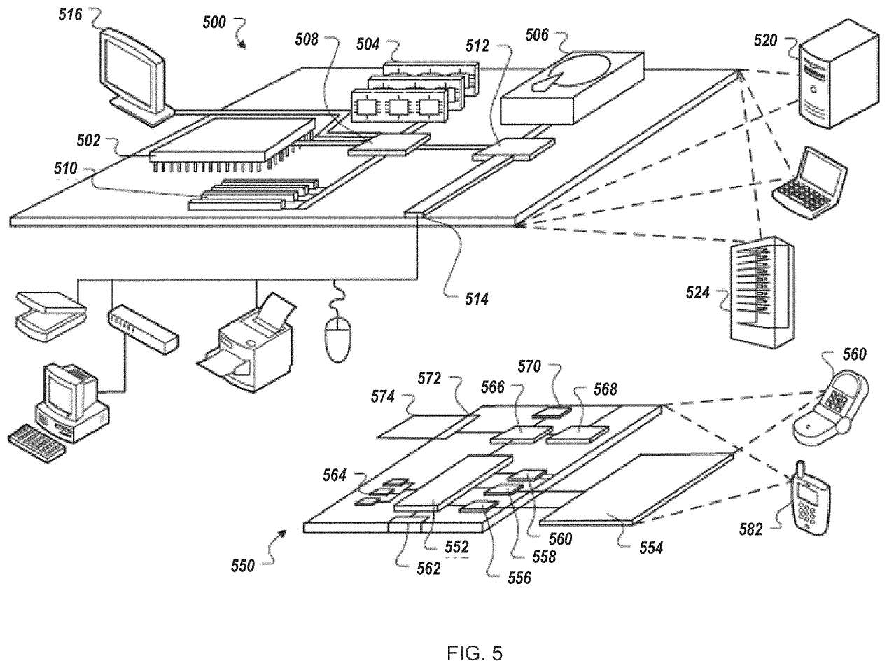

FIG. 5 shows an example of a computing device 500 and a mobile computing device 550 that can be used in the methods and systems described herein. The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk. The storage device 506 is capable of providing mass storage for the computing device 500. In some implementations, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 502), perform one or more methods, such as those described above. The instructions can also be stored by one or more storage devices such as computer- or machine-readable mediums (for example, the memory 504, the storage device 506, or memory on the processor 502).

The high-speed interface 508 manages bandwidth-intensive operations for the computing device 500, while the low-speed interface 512 manages lower bandwidth-intensive operations. Such allocation of functions is an example only. In some implementations, the high-speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device (not shown), such as a mobile computing device 550. Each of such devices may contain one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provided as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 552), perform one or more methods, such as those described above. The instructions can also be stored by one or more storage devices, such as one or more computer- or machine-readable mediums (for example, the memory 564, the expansion memory 574, or memory on the processor 552). In some implementations, the instructions can be received in a propagated signal, for example, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 568 using a radio-frequency. In addition, short-range communication may occur, such as using a Bluetooth®, Wi-Fi™, or other such transceiver (not shown). In addition, a GPS (Global Positioning System) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in the figure. For example, it may be implemented as a cellular telephone 580. It may also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device ((e.g., a LCD (liquid crystal display), LED (light emitting diode), or OLED (organic light emitting diode) for displaying information to the user and a data entry interface (e.g., touch screen, keyboard, touch pad, mouse) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including speech or gestures.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

1.-29. (canceled)

30. A system for providing personalized prognostic profiles, comprising:
at least one memory operable to store a reference database associated with a plurality of individuals, the reference database comprising a combination of time-independent and time-dependent data items associated with each of the plurality of individuals;
a processor communicatively coupled to the at least one memory, the processor being operable to:
(A) receive, over a network, from a client computing device, time-independent and time-dependent data items associated with a person of interest,
wherein each time-dependent data item (1) associated with the person of interest, and (2) located in the reference database is linked to a corresponding time point or time interval;
(B) receive, over the network, from the client computing device, a first request to generate a personalized prognostic profile corresponding to the person of interest, the first request comprising:
(1) an outcome of interest comprising a member selected from the group consisting of mortality, return to work, social outcome and financial outcome;
(2) a display interval indicating the time interval that will be covered by the personalized prognostic profile;
(3) one or more time intervals or time points of interest;
(4) a treatment of interest; and
(5) a set of one or more forced match variables each comprising a clinical or demographic attribute of the person of interest, wherein the set of forced match variables is used to define a subset of the plurality of individuals, each of whom has clinical and/or demographic attributes deemed to match each of the set of forced match variables, wherein each of the forced match variables has an associated range of values, wherein the value may be selected by the person of interest;
(C) generate, in real time, a personalized prognostic profile corresponding to the person of interest comprising one or more widgets that are customizable based on a personal background and personal experience of the person of interest, the personalized prognostic profile comprising: (i) a personalized prognostic graph showing historical outcomes of a matched population selected from the subset of the plurality of individuals matching the forced match variables over the display interval, and (ii) a widget containing identifying information about the person of interest and indicating the forced match variables, the time interval(s) of interest, the treatment of interest, contextual data associated with the person of interest, and the personalized prognostic graph, wherein:
predictions of the outcome of interest are made using one or more outcome predictive models or treatment predictive models, each outcome predictive model or treatment predictive model comprising a member selected from the group consisting of logistic regression, polynomial regression, spline regression, decision tree, neural net, boosted regression, lasso regression, boosted tree, cluster analysis, random forest, and support vector machine methodologies, that are developed using a subset of the matched population that matches the person of interest on the forced match variables; and
the matched population is selected employing an iterative process in which a predictive model of the outcome of interest at each of a plurality of steps is generated, validated, and applied to portions of the subset of the plurality of individuals created at the previous step, the iterative process comprising:
(a) identifying a forced match subset of individuals from among the plurality of individuals associated with the reference database, each of the individuals being associated with a set of parameters corresponding to the forced match variables, wherein the set of parameters of each of the individuals in the forced match subset matches the forced match variable of the person of interest, said forced match variables received in the first request, the forced match variables comprising a variable whose value has been determined by adjusting an adjustable range of values, and wherein each of the individuals is associated with an estimated probability of occurrence of the outcome of interest during one or more user-specified time intervals of interest is within a preset interval of the estimated probability of occurrence of the outcome of interest for the person of interest during that interval;
(b) generating a first outcome predictive model based on the forced match subset that predicts the occurrence of the outcome of interest over one of the one or more user-specified time intervals of interest;
(c) calculating, using the first outcome predictive model, a probability of occurrence of the outcome of interest at a first specified time interval of interest for (1) for the person of interest, and (2) for each of the individuals in the forced match subset;
(d) identifying, from among the individuals in the forced match subset, a first nested subset of individuals with an estimated probability of the occurrence of the outcome of interest within the first specified time interval of interest that is within a given predetermined interval of the probability of occurrence of the outcome of interest for the person of interest;
(e) generating, based on the first nested subset, a treatment predictive model (propensity model) for the receipt of the treatment by each member of the first nested sub set;
(f) applying that propensity model to the person of interest and to all individuals in the first nested subset generated thus far in the process to determine a propensity matched subset, wherein a probability of occurrence of a treatment of interest for each of the individuals in the propensity matched subset is within a given predetermined interval around the estimated probability of the occurrence of the treatment of interest for the person of interest;
(g) creating, from the first nested subset of individuals, a second nested subset of individuals in which each member of the propensity matched subset is associated with a binary variable indicating whether a particular treatment was given;
(h) identifying, based on the binary variable, from the second nested subset, a treatment-true subset and a treatment-false subset; and
(i) designating a nested subset that is the final result of the iterative process as the matched population; and
(D) display the occurrence of the outcome of interest in the matched population over the display interval with the outcome of the treatment-true subset and a treatment-false subset, wherein the personalized prognostic graph comprises a survival curve and indicates the occurrence of the outcome of interest for the individuals in the treatment-true subset and the outcome of interest for the individuals in the treatment-false subset.

31. The system of claim 30, wherein the widget is an interactive widget.

32. The system of claim 30, wherein the forced match variables are specified by the person of interest via an interactive widget, wherein the interactive widget is adapted to a provide a “what if” functionality to cause the personal prognostic graph to display two alternative outcomes based on two alternatives of at least one of the forced match variables

33. The system of claim 30, wherein the system is adapted to conform with a healthcare system comprising a reference database and mortality data.

34. The system of claim 30, wherein step (C)(d) comprises
repeating the process of generating the predictive model for of the outcome of interest and nested subset selection for each of any additional of the time intervals, thereby creating a new predictive model at each repetition to determine the first nested subset.

35. The system of claim 30, comprising one or more supplemental interactive widgets providing further information concerning the person of interest's clinical status, care received, care plans, care preferences, or issues related to illness-related clinical or personal concerns of the person of interest, information related to end-of-life care, palliative care issues, advanced care planning, legal and financial issues, or emotional and spiritual issues.

36. The system of claim 30, wherein the first request comprises a treatment of interest.

37. The system of claim 30, wherein the survival curve is a Kaplan-Meier survival curve accompanied by a widget showing a description of the treatment of interest, its potential benefits, and its typical risks and adverse effects.

38. The system of claim 30, wherein the widget is or comprises an interactive web page that supports an adaptive learning process

39. The system of claim 30, wherein the forced match variables are specified by the person of interest via an interactive widget, wherein the interactive widget is adapted to a cognitive impairment level or educational level of the person of interest.

40. A system for generating online, printable documents (Personalized Treatment Comparisons) to support decision-making about medical therapies, based on an analysis of a database of medical records and other linked data that includes outcomes of treatments under consideration by a user, the system comprising:
at least one memory operable to store a reference database associated with a plurality of individuals, the reference database comprising a combination of data items comprising medical records and other linked data comprising outcomes of treatments, the data items associated with each of a plurality of individuals;
a processor communicatively coupled to the at least one memory, the processor being operable to:
(A) receive, over a network, from a client computing device, one or more data items associated with a person of interest,
(B) receive, over the network, from a client computing device, a first request to generate a Personalized Treatment Comparison corresponding to the person of interest, the first request comprising:
(1) an outcome of interest comprising at least one of a group consisting of mortality, return to work, social outcome and financial outcome;
(2) a display interval indicating the time interval that will be covered by the Personalized Treatment Comparison;
(3) one or more time intervals or time points of interest, wherein the one or more time interval comprise a time interval for a follow-up outcome, and wherein the one or more time points of interest comprise a starting point for historical records, wherein, if two or more time points are specified by the user, the time points are assigned an order of priority,
(4) a treatment of interest, and
(5) exact forced match variables, wherein the exact forced match variables comprise one or more clinical or non-clinical attributes of the person of interest, the one or more clinical or non-clinical attributes comprising at least one of a group consisting of demographic data, social determinants of health, diagnoses, laboratory tests, imaging findings, data from surgical and other procedure notes, and treatments received, the treatments comprising at least one of a group consisting of medications, supplements and OTC medications, and wherein the exact forced match variables comprise one or more values, each value having an adjustable range;
(C) generate, in real time, a Personalized Treatment Comparison corresponding to the person of interest comprising one or more widgets that are customizable based on a personal background and personal experience of the user, the Personalized Treatment Comparison comprising: (i) a personalized prognostic graph showing the historical outcomes of a matched population selected from the subset of the plurality of individuals matching the exact forced match variables over the display interval, and (ii) a first widget containing identifying information about the person of interest, and indicating the exact forced match variables, the time interval(s) of interest, the treatment of interest, and contextual data associated with the person of interest and the personalized prognostic graph, wherein:
predictions of the outcome of interest are made using one or more outcome predictive models or treatment predictive models, each outcome predictive model or treatment predictive model comprising a member selected from the group consisting of logistic regression, polynomial regression, spline regression, decision tree, neural net, boosted regression, lasso regression, boosted tree, cluster analysis, random forest, and support vector machine methodologies, that are developed using a subset of the matched population that matches the person of interest on the exact forced match variables; and
the matched population is selected employing an iterative process in which the predictive model of the outcome of interest at each of a plurality of steps is generated, validated, and applied portions of the subset of the plurality of individuals created at the previous step, the iterative process comprising:
(a) identifying a forced match subset of individuals from among the plurality of individuals associated with the reference database, each of the individuals being associated with a set of parameters corresponding to the exact forced match variables wherein the set of parameters of each of the individuals in the forced match subset matches the exact forced match variable of the person of interest on the exact forced match variables received in the first request and has either received the treatment of interest, or has received a treatment the person of interest would expect to receive in the absence of the treatment of interest;
(b) generating a first outcome predictive model based on the forced match subset that predicts the occurrence of the outcome of interest over the one of the one or more user-specified time intervals of interest;
(c) calculating, using the first outcome predictive model, a probability of occurrence of the outcome of interest at a first specified time interval of interest for (1) for the person of interest, and (2) for each of the individuals in the forced match subset;
(d) identifying, from among the individuals in the forced match subset, a first nested subset of individuals with an estimated probability of the occurrence of the outcome of interest within the first specified time interval of interest that is within a given predetermined interval of the probability of occurrence of the outcome of interest for the person of interest;
(e) generating, based on the first nested subset, a treatment predictive model (propensity model) for the receipt of the treatment by each member of the first nested subset;
(f) applying that propensity model to the person of interest and to all individuals in the first nested subset generated thus far in the process to determine a propensity matched subset, wherein a probability of occurrence of a treatment of interest for each of the individuals in the propensity matched subset is within a given predetermined interval around the estimated probability of the occurrence of the treatment of interest for the person of interest;
(g) creating, from the first nested subset of individuals, a second nested subset of individuals in which each member of the propensity matched subset is associated with a binary variable indicating whether a particular treatment was given;
(h) identifying, based on the binary variable, from the second nested subset a treatment-true subset and a treatment-false subset; and
(i) designating a nested subset that is the final result of the iterative process as the matched population;
(D) display the occurrence of the outcome of interest in the matched population over the display interval with the outcome of the treatment-true subset and a treatment-false subset, wherein the personalized prognostic graph comprises a survival curve and indicates the occurrence of the outcome of interest for the individuals in the treatment-true subset and the outcome of interest for the individuals in the treatment-false subset; and
(E) display a group of interactive widgets alongside the personalized prognostic graph, each of the interactive widgets being customizable in one or more parameters, the parameters comprising at least one of a group consisting of language, educational level, numeracy, and medical knowledge of the user, the interactive widgets comprising
a widget that displays the user's specifications and how the specifications were implemented in generating the personalized prognostic graph; and
a widget that provides data-driven guidance for a clinical or non-clinical decision, the decision comprising pain management or advance directives for end-of-life care.

41. The system of claim 40, wherein the first widget is an interactive widget.

42. The system of claim 40, wherein the exact forced match variables are specified by the person of interest via an interactive widget, wherein the widget is adapted to a provide a “what if” functionality to cause the personal prognostic graph to display two alternative outcomes based on two alternatives of at least one of the exact forced match variables.

43. The system of claim 40, wherein the system is adapted to conform with a healthcare system comprising a reference database and mortality data.

44. The system of claim 40, wherein step (C)(d) comprises
repeating the process of generating the predictive model for of the outcome of interest and nested subset selection for each of any additional of the time intervals, thereby creating a new predictive model at each repetition to determine the first nested subset.

45. The system of claim 40, comprising one or more supplemental interactive widgets providing further information concerning the person of interest's clinical status, care received, care plans, care preferences, or issues related to illness-related clinical or personal concerns of the person of interest, information related to end-of-life care, palliative care issues, advanced care planning, legal and financial issues, or emotional and spiritual issues.

46. The system of claim 40, wherein the survival curve is a Kaplan-Meier survival curve accompanied by a widget showing a description of the treatment of interest, its potential benefits, and its typical risks and adverse effects.

47. The system of claim 40, wherein the treatment the person of interest would expect to receive in the absence of the treatment of interest comprises:
(a) a current standard of non-surgical treatment of the person of interest's condition, if the treatment under consideration is surgery, or
(b) palliative care only, if the treatment under consideration is aggressive treatment of all medical conditions for a person of interest with a terminal prognosis.

48. The system of claim 40, wherein the first widget is or comprises an interactive web page that supports an adaptive learning process.

49. The system of claim 40, wherein the exact forced match variables are specified by the person of interest via an interactive widget, wherein the interactive widget is adapted to a cognitive impairment level or educational level of the user.
